(19) United States
(12) Patent Application Publication
(10) Pub. No.: US 2012/0028990 A1
(43) Pub. Date: Feb. 2, 2012

(54) 3 ARYL OR HETEROARYL-SUBSTITUTED INDOLE DERIVATIVE

(75) Inventors: Takashi Fujimura, Aomori (JP); Hiroyuki Kishino, Tokyo (JP); Takashi Mizutani, Ibaraki (JP); Shunji Sakuraba, Ibaraki (JP); Takahide Sasaki, Kanagawa (JP); Nagaaki Sato, Saitama (JP)

(73) Assignee: MSD K.K

(21) Appl. No.: 13/260,746

(22) PCT Filed: Apr. 14, 2010
(86) PCT No.: PCT/JP2010/056965

§ 371 Date: Oct. 18, 2011

(51) Int. Cl. A61K 31/404; A61K 31/4439; A61K 31/4725; A61P 3/04; C07D 403/12; A61K 31/496; C07D 417/14; C07D 409/14; C07D 405/14; A61K 31/454

(52) U.S. Cl. 514/254.02; 546/278.1; 514/339; 548/466; 514/414; 546/143; 514/310; 546/201; 514/323; 544/373; 514/254.09; 544/369

(57) ABSTRACT

A 3-aryl or heteroaryl-substituted indole derivative which is effective as a preventive or remedy for various diseases is provided. [Means for Resolution] A compound represented by a formula (I) or a pharmaceutically acceptable salt thereof is provided. In the formula, R1 represents a hydrogen atom, C1-6 alkyl et al; R2 represents a hydrogen atom, C1-6 alkyl et al; Z represents aryl or heteroaryl (with the proviso that imidazolyl is excluded); and W represents a group represented by the formula (w-1) or (w-2). In the formula, R3 represents C1-6 alkyl et al; Yl and Y2 both represent a hydrogen atom or Y1 and Y2 together form —CH2-CH2-; Q represents CH or N; Ar represents phenyl, furyl et al; R4 and R5 each independently represent a hydrogen atom or C1-6 alkyl or R4 and R5 together with the nitrogen atom form pyrrolidine et al.

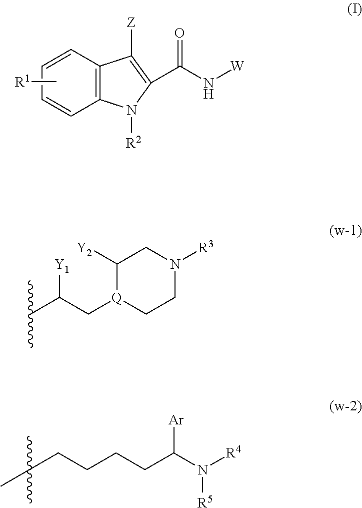

### TECHNICAL FIELD

The present invention relates to a 3-substituted indole derivative useful as medicines. The compound acts as an antagonist of human QRFP receptor (GPR103) and is useful as a preventive or remedy for obesity and the like.

### BACKGROUND ART

QRFP43 is a peptide comprising 43 amino acid residues, and it was reported in 2003 that QRFP43 is an endogenous ligand for QRFP43 receptor (GPR103) through bioinformatics and reverse pharmacological analysis, and thereafter in 2006, it was isolated for the first time from rat brain (see, for example, Non-patent document 1). Further, it is described that a QRFP43 analogue, for example, 26RFa binds to QRFP43 receptor and shows a similar activity to that of QRFP43 (see, for example, Patent documents 1 and 2). QRFP43 is largely expressed in the central nervous system, particularly in the hypothalamus and controls various functions in the living body. That is, QRFP43 functions as an orexigenic substance in the central nervous system and significantly promotes fat accumulation via the secretion of various hormones or nervous system activity. It is known that repeated intracerebroventricular administration of QRFP43 induces obesity and insulin resistance based on these activities. Further, it also participates in secretion of hormones such as hypothalamic hormone and pituitary hormone.

The function of QRFP43 or 26RFa is exhibited when it binds to QRFP43 receptor (GPR103) present in the central or peripheral nervous system. Therefore, if the binding of QRFP43 or 26RFa to QRFP43 receptor (GPR103) is inhibited, the expression of activity of QRFP43 or 26RFa can be prevented.

As a compound related to the indole derivative according to the invention, for example, in WO 2008/119741 (Patent document 3), a compound having an imidazolyl group at the 2-position of an indole ring has been disclosed. However, the compound described in this document is an inhibitor of MDM2 and MDM4 and there is a description that the compound is useful for the prevention or treatment of proliferative diseases such as cancer, and therefore, the compound is different in use from the compound of the present invention. Further, in JP-A-62-234065 (Patent document 4), an indole derivative has been disclosed, however, the Z moiety of the compound of the present invention is different. Also, the compound described in this reference document is used for dementia, cerebrovascular disorder and the like, which is different from the use of the present invention.

[Non-patent document 1] Proceedings of the National Academy of Sciences of the United States of America, Vol. 103, pp. 7438-7443, (2006)

[Patent document 1] WO 01/16316

[Patent document 2] WO 05/65702

[Patent document 3] WO 2008/119741

[Patent document 4] JP-A-62-234065

### DISCLOSURE OF THE INVENTION

### Problems that the Invention is to Solve

If the binding of QRFP43 or 26RFa to QRFP43 receptor (GPR103) is inhibited, the expression of activity of QRFP43 or 26RFa can be prevented. Therefore, it can be expected that a substance which antagonizes the binding of QRFP43 or 26RFa to QRFP43 receptor (GPR103) has usefulness in the prevention or treatment of various diseases related to QRFP43 or 26RFa, for example, cardiovascular diseases such as hypertension, arteriosclerosis, renal diseases, heart diseases and vasospasm; bulimia; and metabolic diseases such as obesity, diabetes, abnormal hormone secretion, hypercholesterolemia, hyperlipidemia, gout and fatty liver. Further, the substance can be used as a preventive or remedy for pain, circadian rhythm disorder, atherosclerosis, obesity-related gastroesophageal reflux, obesity hypoventilation syndrome (Pickwickian syndrome), hypertriglyceridemia, low HDL cholesterolemia, cardiovascular diseases such as coronary heart diseases (CHD), peripheral vascular diseases and sudden death, pain, osteoporosis-related diseases, low back pain, anesthetic hypersensitivity and the like.

Accordingly, an object of the invention is to provide an antagonist of QRFP43 receptor (GPR103) which is useful as a preventive or remedy for the above-mentioned diseases.

### Means for Solving the Problems

As a result of intensive studies, the present inventors have found that indole derivatives which are substituted by aryl or heteroaryl at the 3-position of indole and further have an alkylaminocarbonyl substituent at the 2-position have an excellent antagonistic activity against human QRFP43 receptor (GPR103), and have completed the present invention.

That is, the invention provides:

(1) A compound represented by a formula (I) or a pharmaceutically acceptable salt thereof:

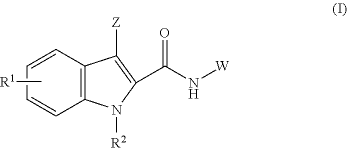

[wherein R¹ represents a hydrogen atom, halogen, C₁₋₆ alkyl, halo-C₁₋₆ alkyl, C₁₋₆ alkyloxy or halo-C₁₋₆ alkyloxy;

R² represents a hydrogen atom, C₁₋₆ alkyl or halo-C₁₋₆ alkyl;

W represents a group represented by the formula (w-1) or (w-2):

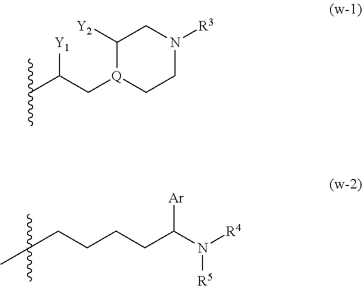

(wherein R³ represents C₁₋₆ alkyl which may be substituted by a substituent selected from a group consisting of halogen, hydroxy and phenyl;

Y₁ and Y₂ both represent a hydrogen atom or Y₁ and Y₂ together form —CH₂—CH₂—;

Q represents CH or N;

Ar is selected from a group consisting of phenyl, furyl, pyridyl, pyrimidyl, (1-methyl-1H-pyrazol)-3-yl and (1-methyl-1H-pyrazol)-4-yl;

R⁴ and R⁵ each independently represent a hydrogen atom or C₁₋₆ alkyl or R⁴ and R⁵, together with the nitrogen atom to which they are attached form a pyrrolidine ring or a piperidine ring);

Z represents aryl or heteroaryl (with the proviso that imidazolyl is excluded), wherein the aryl or heteroaryl may be substituted by one to three substituents selected from a group consisting of halogen, C₁₋₆ alkyl, halo-C₁₋₆ alkyl, C₁₋₆ alkyloxy, halo-C₁₋₆ alkyloxy, hydroxy, amino, mono-C₁₋₆ alkylamino, di-C₁₋₆ alkylamino, C₁₋₆ alkyloxycarbonyl, C₁₋₆ alkyloxycarbonylamino, C₁₋₆ alkyloxycarbonyl(C₁₋₆ alkyl) amino, C₁₋₆ alkylcarbonyl, C₁₋₆ alkylcarbonyloxy, C₁₋₆ alkylcarbonylamino, C₁₋₆ alkylcarbonyl(C₁₋₆ alkyl)amino, carbamoyl, mono-C₃ alkylcarbamoyl, di-C₁₋₆ alkylcarbamoyl, carbamoylamino, mono-C₁₋₆ alkylcarbamoylamino, di-C₁₋₆ alkylcarbamoylamino, mono-C₁₋₆ alkylcarbamoyl(C₁₋₆ alkyl) amino, di-C₁₋₆ alkylcarbamoyl(C₁₋₆ alkyl)amino, carbamoyloxy, mono-C₁₋₆ alkylcarbamoyloxy, di-C₁₋₆ alkylcarbamoyloxy, C₁₋₆ alkylsulfonyl, C₁₋₆ alkylsulfonylamino, C₁₋₆ alkylsulfonyl (C₁₋₆ alkyl)amino, sulfamoyl, mono-C₁₋₆alkylsulfamoyl, di-C₁₋₆alkylsulfamoyl, sulfamoylamino, mono-C₁₋₆ alkylsulfamoylamino, di-C₁₋₆ alkylsulfamoylamino, mono-C₁₋₆ alkylsulfamoyl(C₁₋₆ alkyl)amino and di-C₁₋₆ alkylsulfamoyl (C₁₋₆ alkyl)amino];

(2) A compound represented by a formula (I-1) or a pharmaceutically acceptable salt thereof:

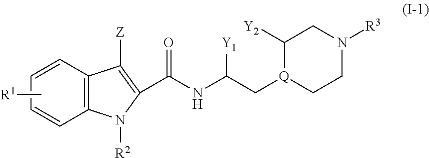

[wherein R¹, R², R³, Y₁, Y₂, Q and Z have the same meanings as described above];

(3) A compound represented by a formula (I-2) or a pharmaceutically acceptable salt thereof:

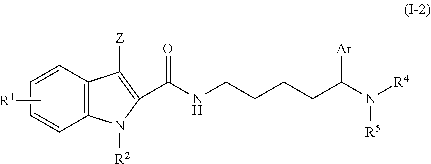

[wherein R¹, R², R⁴, R⁵, Z and Ar have the same meanings as described above];

(4) A pharmaceutical composition comprising the compound or the pharmaceutically acceptable salt thereof according to the above (1) to (3) and a pharmaceutically acceptable carrier; and

(5) A preventive or remedy for obesity comprising the compound or the pharmaceutically acceptable salt thereof according to the above (1) to (3) as an active ingredient.

### BEST MODE FOR CARRYING OUT THE INVENTION

Hereinafter, the invention will be described in more detail.

Examples of the “halogen” include fluoro, chloro, bromo and iodo.

The “C₁₋₆ alkyl” includes linear alkyl having 1 to 6 carbon atoms and branched alkyl having 3 to 6 carbon atoms, and specific examples thereof include methyl, ethyl, n-propyl, isopropyl, n-butyl, isobutyl, sec-butyl, tert-butyl, n-pentyl, isopentyl, neopentyl, tert-amyl, 2-propyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

The “halo-C₁₋₆ alkyl” includes C₁₋₆ alkyl in which some or all of the hydrogen atoms of C₁₋₆ alkyl are substituted by halogen, and examples thereof include fluoromethyl, difluoromethyl, trifluoromethyl, 2-fluoroethyl and 1,2-difluoroethyl.

The “C₁₋₆ alkyloxy” includes a group in which C₁₋₆ alkyl is bound to an oxygen atom, and specific examples thereof include methoxy, ethoxy, n-propyloxy, isopropyloxy, n-butyloxy, isobutyloxy, tert-butyloxy and n-pentyloxy.

The “halo-C₁₋₆ alkyloxy” includes a group in which halo-C₁₋₆ alkyl is bound to an oxygen atom, and specific examples thereof include fluoromethoxy, chloromethoxy, difluoromethoxy, dichloromethoxy, trifluoromethoxy, trichloromethoxy, 2-fluoroethoxy and 1,2-difluoroethoxy.

The “mono-C₁₋₆ alkylamino” is a group in which one of the hydrogen atoms of amino (—NH₂) is replaced by a C₁₋₆ alkyl group, and specific examples thereof include methylamino, ethylamino, n-propylamino, isopropylamino, n-butylamino, sec-butylamino and tert-butylamino.

The “di-C₁₋₆ alkylamino” is a group in which two hydrogen atoms of amino are replaced by C₁₋₆ alkyl, and specific examples thereof include dimethylamino, diethylamino, ethylmethylamino, di(n-propyl)amino, methyl(n-propyl)amino and diisopropylamino.

The “C₁₋₆ alkyloxycarbonyl” is a group in which C₁₋₆ alkyloxy is bound to carbonyl (—CO—) and includes alkyloxycarbonyl having 1 to 6 carbon atoms. Specific examples thereof include methoxycarbonyl, ethoxycarbonyl, n-propyloxycarbonyl, isopropyloxycarbonyl, n-butoxycarbonyl, isobutoxycarbonyl, tert-butoxycarbonyl and n-pentyloxycarbonyl.

The “C₁₋₆ alkyloxycarbonylamino” is a group in which one of the hydrogen atoms of amino is replaced by C₁₋₆ alkyloxycarbonyl and includes alkyloxycarbonylamino having 1 to 6 carbon atoms. Specific examples thereof include methoxycarbonylamino, ethoxycarbonylamino, n-propyloxycarbonylamino, isopropyloxycarbonylamino, n-butoxycarbonylamino, isobutoxycarbonylamino, tert-butoxycarbonylamino and n-pentyloxycarbonylamino.

The “C₁₋₆ alkyloxycarbonyl(C₁₋₆alkyl)amino” is a group in which C₁₋₆ alkyloxycarbonyl is bound to the nitrogen atom of mono-C₁₋₆ alkylamino instead of a hydrogen atom, and specific examples thereof include methoxycarbonyl (methyl)amino, ethoxycarbonyl(methyl)amino and n-propyloxycarbonyl(methyl)amino. The “C₁₋₆alkylcarbonyl” is a group in which C₁₋₆ alkyl is bound to carbonyl and includes alkylcarbonyl having 1 to 6 carbon atoms. Specific examples thereof include acetyl, propionyl, butyryl, isobutyryl, valeryl, isovaleryl and pivaloyl.

The “C₁₋₆ alkylcarbonyloxy” is a group in which C₁₋₆ alkylcarbonyl is bound to an oxygen atom, and specific examples thereof include acetoxy, propionyloxy, valeryloxy, isovaleryloxy and pivaloyloxy.

The “C₁₋₆ alkylcarbonylamino” is a group in which one of the hydrogen atoms of an amino group is replaced by C₁₋₆ alkylcarbonyl, and specific examples thereof include acetylamino, propionylamino, isobutyrylamino, valerylamino, isovalerylamino and pivaloylamino.

The “C₁₋₆ alkylcarbonyl(C₁₋₆ alkyl)amino” is a group in which a hydrogen atom attached to the nitrogen atom of mono-C₁₋₆ alkylamino is replaced by C₁₋₆ alkylcarbonyl, and specific examples thereof include methylcarbonyl(methyl)amino, ethylcarbonyl(methyl)amino and n-propylcarbonyl(methyl)amino.

The “mono-C₁₋₆ alkylcarbamoyl” is a group in which one of the hydrogen atoms of carbamoyl (—CONH₂) is replaced by C₁₋₆ alkyl, and specific examples thereof include methylcarbamoyl, ethylcarbamoyl, n-propylcarbamoyl, isopropylcarbamoyl, n-butylcarbamoyl, sec-butylcarbamoyl and tert-butylcarbamoyl.

The “di-C₁₋₆ alkylcarbamoyl” is a group in which two hydrogen atoms of carbamoyl are replaced by C₁₋₆ alkyl, and specific examples thereof include dimethylcarbamoyl, diethylcarbamoyl, ethylmethylcarbamoyl, di(n-propyl)carbamoyl, methyl(n-propyl)carbamoyl and diisopropylcarbamoyl.

The “mono-C₁₋₆ alkylcarbamoylamino” is a group in which one of the hydrogen atoms of amino is replaced by mono-C₁₋₆ alkylcarbamoyl, and specific examples thereof include methylcarbamoylamino, ethylcarbamoylamino, n-propylcarbamoylamino, isopropylcarbamoylamino, n-butylcarbamoylamino, sec-butylcarbamoylamino and tert-butylcarbamoylamino.

The “di-C₁₋₆ alkylcarbamoylamino” is a group in which one of the hydrogen atoms of amino is replaced by di-C₁₋₆ alkylcarbamoyl, and specific examples thereof include dimethylcarbamoylamino, diethylcarbamoylamino, di(n-propyl)carbamoylamino, diisopropylcarbamoylamino, di(n-butyl)carbamoylamino, di(sec-butyl)carbamoylamino and di(tert-butyl)carbamoylamino.

The “mono-C₁₋₆ alkylcarbamoyl(C₁₋₆alkyl)amino” is a group in which a hydrogen atom attached to the nitrogen atom of mono-C₁₋₆ alkylamino is replaced by mono-C₁₋₆ alkylcarbamoyl, and specific examples thereof include monomethylcarbamoyl(methyl)amino, monoethylcarbamoyl (methyl)amino and [mono(n-propyl)carbamoyl](methyl)amino.

The “di-C₁₋₆ alkylcarbamoyl(C₁₋₆alkyl)amino” is a group in which a hydrogen atom attached to the nitrogen atom of mono-C₁₋₆ alkylamino is replaced by di-C₁₋₆ alkylcarbamoyl, and specific examples thereof include dimethylcarbamoyl(methyl)amino, diethylcarbamoyl(methyl) amino and [di(n-propyl)carbamoyl](methyl)amino.

The “mono-C₁₋₆ alkylcarbamoyloxy” is a group in which mono-C₁₋₆ alkylcarbamoyl is bound to an oxygen atom, and specific examples thereof include methylcarbamoyloxy, ethylcarbamoyloxy, n-propylcarbamoyloxy, isopropylcarbamoyloxy, n-butylcarbamoyloxy, sec-butylcarbamoyloxy and tert-butylcarbamoyloxy.

The “di-C₁₋₆ alkylcarbamoyloxy” is a group in which di-C₁₋₆ alkylcarbamoyl is bound to an oxygen atom, and specific examples thereof include dimethylcarbamoyloxy, diethylcarbamoyloxy, ethylmethylcarbamoyloxy, di(n-propyl)carbamoyloxy, methyl(n-propyl)carbamoyloxy and diisopropylcarbamoyloxy.

The “C₁₋₆ alkylsulfonyl” is a group in which C₁₋₆ alkyl is bound to sulfonyl (—SO₂—), and specific examples thereof include methanesulfonyl, ethanesulfonyl, n-propanesulfonyl, isopropanesulfonyl, n-butanesulfonyl, sec-butanesulfonyl and tert-butanesulfonyl.

The “C₁₋₆ alkylsulfonylamino” is a group in which one of the hydrogen atoms of amino is replaced by C₁₋₆ alkylsulfonyl, and specific examples thereof include methanesulfonylamino, ethanesulfonylamino, n-propanesulfonylamino, isopropanesulfonylamino, n-butanesulfonylamino, sec-butanesulfonylamino and tert-butanesulfonylamino.

The “C₁₋₆ alkylsulfonyl(C₁₋₆ alkylamino” is a group in which a hydrogen atom attached to the nitrogen atom of mono-C₁₋₆ alkylamino is replaced by C₁₋₆ alkylsulfonyl, and specific examples thereof include methanesulfonyl(methyl)amino, ethanesulfonyl(methyl) amino, n-propanesulfonyl(methyl)amino and isopropanesulfonyl(methyl)amino.

The “mono-C₁₋₆ alkylsulfamoyl” is a group in which one of the hydrogen atoms of sulfamoyl (—SO₂NH₂) is replaced by C₁₋₆ alkyl, and specific examples thereof include monomethylsulfamoyl, monoethylsulfamoyl, mono(n-propyl) sulfamoyl, monoisopropylsulfamoyl, mono(n-butyl) sulfamoyl, mono(sec-butyl)sulfamoyl and mono(tert-butyl) sulfamoyl.

The “di-C₁₋₆ alkylsulfamoyl” is a group in which two hydrogen atoms of sulfamoyl are replaced by C₁₋₆ alkyl, and specific examples thereof include dimethylsulfamoyl, diethylsulfamoyl, di(n-propyl)sulfamoyl, diisopropylsulfamoyl, di(n-butyl)sulfamoyl, di(sec-butyl)sulfamoyl and di(tert-butyl)sulfamoyl.

The “mono-C₁₋₆alkylsulfamoylamino” is a group in which one of the hydrogen atoms of amino is replaced by mono-C₁₋₆ alkylsulfamoyl, and specific examples thereof include (monomethylsulfamoyl)amino, (monoethylsulfamoyl)amino, [mono(n-propyl)sulfamoyl]amino, (monoisopropylsulfamoyl)amino, [mono(n-butyl)sulfamoyl]amino, [mono(sec-butyl)sulfamoyl]amino and [mono(tert-butyl)sulfamoyl]amino.

The “(di-C₁₋₆ alkylsulfamoyl)amino” is a group in which one of the hydrogen atoms of amino is replaced by di-C₁₋₆ alkylsulfamoyl, and specific examples thereof include (dimethylsulfamoyl)amino, (diethylsulfamoyl)amino, (ethylmethylsulfamoyl)amino, [di(n-propyl)sulfamoyl]amino, [methyl(n-propyl)sulfamoyl]amino and (diisopropylsulfamoyl)amino.

The “mono-C₁₋₆ alkylsulfamoyl(C₁₋₆alkyl)amino” is a group in which a hydrogen atom attached to the nitrogen atom of mono-C₁₋₆ alkylamino is replaced by mono-C₁₋₆ alkylsulfamoyl, and specific examples thereof include monomethylsulfamoyl(methyl)amino, monoethylsulfamoyl (methyl)amino and [mono(n-propyl)sulfamoyl](methyl)amino.

The “di-C₁₋₆alkylsulfamoyl(C₁₋₆ alkyl)amino” is a group in which a hydrogen atom attached to the nitrogen atom of mono-C₁₋₆ alkylamino is replaced by di-C₁₋₆ alkylsulfamoyl, and specific examples thereof include dimethylsulfamoyl(methyl)amino, diethylsulfamoyl(methyl) amino and [di(n-propyl)sulfamoyl](methyl)amino.

Examples of the “aryl” include phenyl and naphthyl.

The “heteroaryl” means 5- or 6-membered monocyclic heteroaryl having one or more, preferably one or two heteroatoms which are the same or different and are selected from the group consisting of an oxygen atom, a nitrogen atom and a sulfur atom, or condensed cyclic heteroaryl formed by condensation of the monocyclic heteroaryl with the above-mentioned aryl, or condensation of the monocyclic heteroaryl groups which are the same or different. Examples thereof include pyrrolyl, furyl, thienyl, pyrazolyl, thiazolyl, isothiazolyl, oxazolyl, isoxazolyl, triazolyl, tetrazolyl, oxadiazolyl, 1,2,3-thiadiazolyl, 1,2,4-thiadiazolyl, 1,3,4-thiadiazolyl, pyridyl, pyrazinyl, pyrimidinyl, pyridazinyl, 1,2,4-triazinyl, 1,3,5-triazinyl, indolyl, benzofuranyl, benzothienyl, benzimidazolyl, benzopyrazolyl, benzoxazolyl, benzisoxazolyl, benzothiazolyl, benzisothiazolyl, indazolyl, purinyl, quinolyl, isoquinolyl, phthalazinyl, naphthyridinyl, quinoxalinyl, quinazolinyl, cinnolinyl, pteridinyl and pyrido[3,2-b]pyridyl.

The “pharmaceutically acceptable salt” of the derivative represented by the formula (I) includes commonly used pharmaceutically acceptable salts, and an acid addition salt at an amine moiety of the compound of the formula (I) or an acid addition salt at a nitrogen-containing heterocyclic ring, or in the case where the compound of the formula (I) has an acidic substituent, a base addition salt at the acidic substituent or the like can be exemplified.

Examples of the acid addition salt include inorganic acid salts such as hydrochlorides, sulfates, nitrates, phosphates and perchlorates; organic acid salts such as maleates, fumarates, tartrates, citrates, ascorbates and trifluoroacetates; and sulfonates such as methanesulfonates, isothiocyanates, benzenesulfonates and p-toluenesulfonates.

Examples of the base addition salt include alkali metal salts such as sodium salts and potassium salts; alkaline earth metal salts such as calcium salts and magnesium salts; ammonium salts; and organic amine salts such as trimethylamine salts, triethylamine salts, dicyclohexylamine salts, ethanolamine salts, diethanolamine salts, triethanolamine salts, procaine salts and N,N′-dibenzylethylenediamine salts.

For illustrating the derivatives of the invention more specifically, each symbols to be used in the formula (I) will be described with reference to specific examples thereof below.

R¹ represents a hydrogen atom, halogen, C₁₋₆ alkyl, halo-C₁₋₆ alkyl, C₁₋₆ alkyloxy or halo-C₁₋₆ alkyloxy.

As R¹, specifically, a hydrogen atom; halogen such as fluoro, chloro, bromo and iodo; C₁₋₆ alkyl such as methyl, ethyl, n-propyl, isopropyl, n-butyl and t-butyl; halo-C₁₋₆ alkyl such as chloromethyl, trichloromethyl, fluoromethyl, trifluoromethyl, chloroethyl and fluoroethyl; C₁₋₆ alkyloxy such as methoxy, ethoxy, n-propyloxy and isopropyloxy; and halo-C₁₋₆ alkyloxy such as chloromethoxy, trichloromethoxy, fluoromethoxy, trifluoromethoxy, fluoroethoxy and fluoropropyloxy are exemplified, and preferably, a hydrogen atom or chloro is recommended.

R² represents a hydrogen atom, C₁₋₆ alkyl or halo-C₁₋₆ alkyl.

As R², specifically, a hydrogen atom; C₁₋₆ alkyl such as methyl, ethyl, n-propyl, isopropyl, n-butyl and t-butyl; and halo-C₁₋₆ alkyl such as chloromethyl, trichloromethyl, fluoromethyl, trifluoromethyl, chloroethyl and fluoroethyl are exemplified, and preferably, a hydrogen atom or methyl is recommended.

W represents a group represented by the formula (w-1) or (w-2).

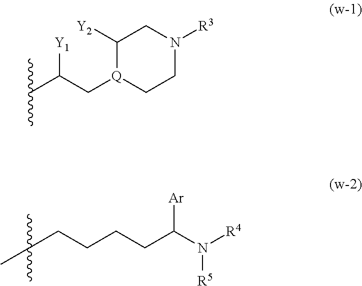

In the formula, R³ represents C₁₋₆ alkyl which may be substituted by a substituent selected from the group consisting of halogen, hydroxy and phenyl;

Y₁ and Y₂ both represent a hydrogen atom or Y₁ and Y₂ together form —CH₂—CH₂—;

Q represents CH or N;

Ar is selected from the group consisting of phenyl, furyl, pyridyl, pyrimidyl, (1-methyl-1H-pyrazol)-3-yl and (1-methyl-1H-pyrazol)-4-yl; and

R⁴ and R⁵ each independently represent a hydrogen atom or C₁₋₆ alkyl or R⁴ and R⁵, together with the nitrogen atom to which they are attached form a pyrrolidine ring or a piperidine ring.

As R³, specifically, C₁₋₆ alkyl such as methyl, ethyl, n-propyl, isopropyl, n-butyl and t-butyl; halo-C₁₋₆ alkyl such as chloromethyl, trichloromethyl, fluoromethyl, trifluoromethyl, chloroethyl and fluoroethyl; hydroxy-substituted C₁₋₆ alkyl such as hydroxymethyl and hydroxyethyl; and phenyl-substituted C₁₋₆ alkyl such as benzyl are exemplified, and preferably, C₁₋₆ alkyl, particularly methyl is recommended.

Y₁ and Y₂ both represent a hydrogen atom or Y₁ and Y₂ together form —CH₂—CH₂—. As Y₁ and Y₂, preferably, a hydrogen atom is recommended for both.

Q represents CH or N, and preferably, N is recommended.

Ar represents phenyl, furyl, pyridyl, pyrimidyl, (1-methyl-1H-pyrazol)-3-yl or (1-methyl-1H-pyrazol)-4-yl, and more specifically, phenyl, 2-furyl, 3-furyl, 2-pyridyl, 3-pyridyl, 4-pyridyl, 2,5-pyrimidyl, (1-methyl-1H-pyrazol)-3-yl and (1-methyl-1H-pyrazol)-4-yl are exemplified, and preferably, furyl (particularly 2-furyl) or pyridyl (particularly 2-pyridyl) is recommended.

As R⁴ and R⁵, preferably, a hydrogen atom, methyl, ethyl, n-propyl or isopropyl is recommended, or it is recommended that R⁴ and R⁵ be combined together with the nitrogen atom to which they are attached to form pyrrolidine.

Among the groups represented by W, particularly, a group represented by the formula (w-1) is preferred.

Z represents aryl or heteroaryl (with the proviso that imidazolyl is excluded), wherein the aryl or heteroaryl may be substituted by one to three substituents selected from the group consisting of halogen, C₁₋₆ alkyl, halo-C₁₋₆ alkyl, C₁₋₆ alkyloxy, halo-C₁₋₆ alkyloxy, hydroxy, amino, mono-C₁₋₆ alkylamino, alkylamino, C₁₋₆ alkyloxycarbonyl, C₁₋₆ alkyloxycarbonylamino, C₁₋₆ alkyloxycarbonyl(C₁₋₆ alkyl) amino, C₁₋₆ alkylcarbonyl, C₁₋₆ alkylcarbonyloxy, C₁₋₆ alkylcarbonylamino, C₁₋₆ alkylcarbonyl(C₁₋₆ alkyl)amino, carbamoyl, mono-C₁₋₆ alkylcarbamoyl, di-C₁₋₆ alkylcarbamoyl, carbamoylamino, mono-C₁₋₆ alkylcarbamoylamino, di-C₁₋₆ alkylcarbamoylamino, mono-C₁₋₆ alkylcarbamoyl(C₁₋₆ alkyl) amino, di-C₁₋₆alkylcarbamoyl(C₁₋₆alkyl)amino, carbamoyloxy, mono-C₁₋₆ alkylcarbamoyloxy, di-C₁₋₆ alkylcarbamoyloxy, C₁₋₆ alkylsulfonyl, C₁₋₆ alkylsulfonylamino, C₁₋₆ alkylsulfonyl(C₁₋₆ alkyl)amino, sulfamoyl, mono-C₁₋₆ alkylsulfamoyl, di-C₁₋₆ alkylsulfamoyl, sulfamoylamino, mono-C₁₋₆ alkylsulfamoylamino, di-C₁₋₆ alkylsulfamoylamino, mono-C₁₋₆ alkylsulfamoyl(C₁₋₆alkyl)amino and di-C₁₋₆ alkylsulfamoyl (C₁₋₆ alkyl)amino.

As an arbitrary substituent by which the aryl or heteroaryl represented by Z may be substituted, preferably, it is unsubstituted; or halogen such as fluoro or chloro; C₁₋₆ alkyl such as methyl, ethyl or isopropyl; halo-C₁₋₆ alkyl such as fluoromethyl or trifluoromethyl; C₁₋₆ alkyloxy such as methoxy, ethoxy or isopropyloxy; halo-C₁₋₆ alkyloxy such as fluoromethoxy or trifluoromethoxy; or the like is recommended, and particularly, it is unsubstituted or halogen (particularly fluoro) is recommended.

As Z, preferably, phenyl, furyl, thienyl or thiazolyl is recommended.

As the compounds represented by the formula (I), specifically, the following compounds are exemplified.

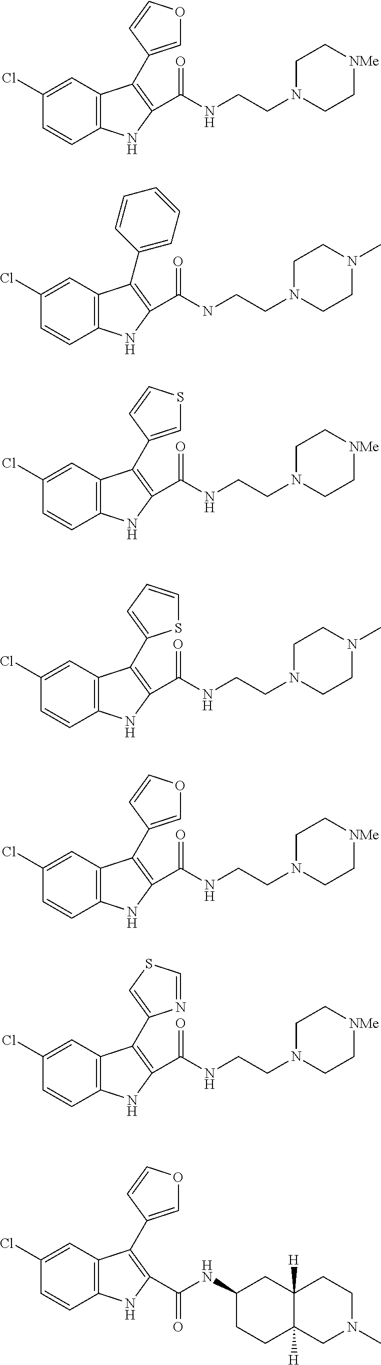

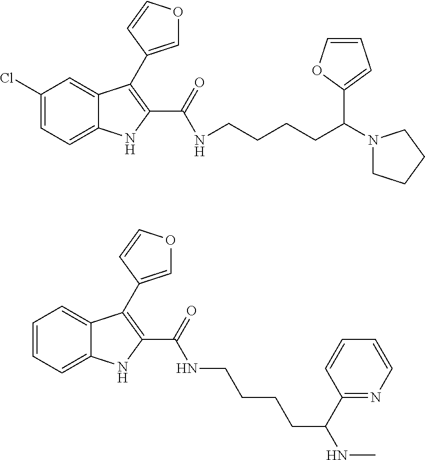

Further, the invention provides the following compound. This compound is also acts as an antagonist of human QRFP receptor (GPR103).

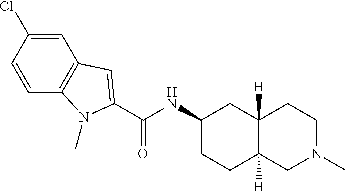

### Method for Producing Compound Represented by Formula (I)

The compound of the invention can be produced by the following method.

### Production Method 1

Production Method 1 is a method for producing a compound represented by a formula (I-1).

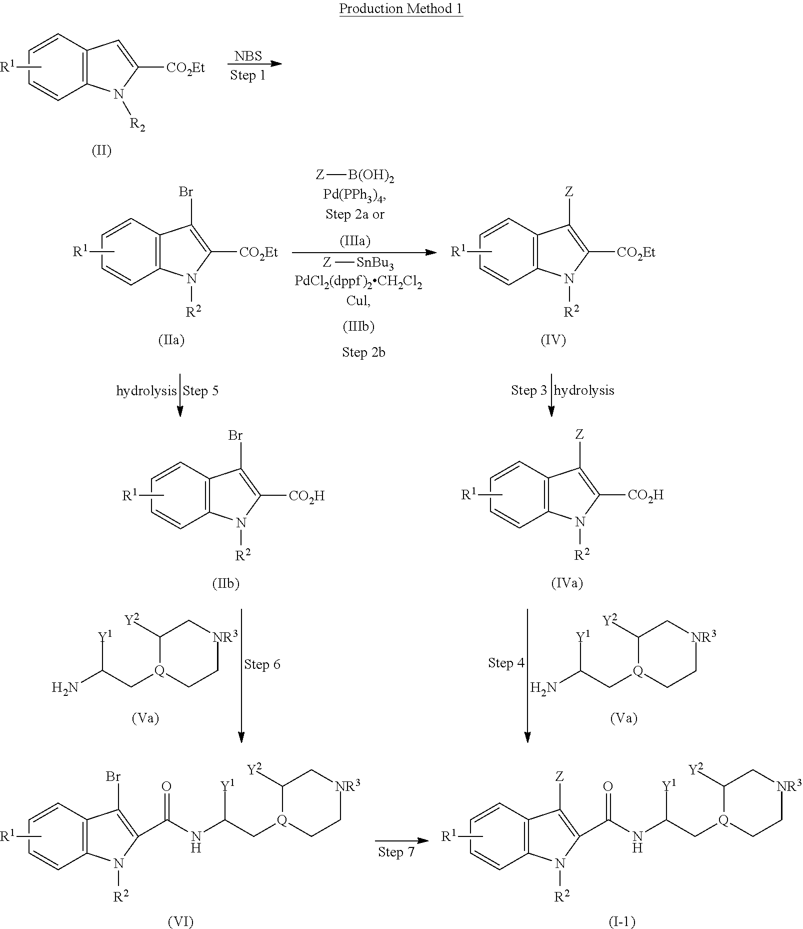

In the formula, each symbol has the same meanings as described above.

### Step 1

A compound represented by the formula (II) is reacted with N-bromosuccinimide (NBS) in an organic solvent, whereby a compound represented by the formula (IIa) is obtained.

As the used amount of NBS, an amount of from 1 to 2 moles per mole of the compound represented by the formula (II) is exemplified, and preferably, 1 to 1.2 moles is recommended.

As the organic solvent, tetrahydrofuran (hereinafter referred to as “THF”), N,N-dimethylformamide (hereinafter referred to as “DMF”), carbon tetrachloride and the like are exemplified.

As the reaction temperature, a temperature of from room temperature to 80° C. is exemplified, preferably, room temperature is recommended, and generally, the reaction is completed in 1 to 24 hours.

Here, as the compound represented by the formula (II), ethyl 5-chloro-1H-indole-2-carboxylate, ethyl 1H-indole-2-carboxylate and the like are exemplified.

### Step 2a

The compound represented by the formula (IIa) is reacted with a compound represented by the formula (IIIc) in an organic solvent in the presence of tetrakis (triphenylphosphine)palladium and a base, whereby a compound represented by the formula (IV) is obtained.

As the used amount of the compound represented by the formula (IIIa), an amount of from 1.0 to 2.0 moles per mole of the compound represented by the formula (IIa) is exemplified, and preferably, 1.2 moles is recommended. As the used amount of tetrakis (triphenylphosphine)palladium, an amount of from 0.05 to 0.2 moles per mole of the compound represented by the formula (IIa) is exemplified, and preferably, 0.1 to 0.2 moles is recommended.

As the base, sodium carbonate, potassium carbonate, sodium hydrogen carbonate, sodium hydroxide and potassium hydroxide are exemplified, and as the used amount of the base, an amount of from 1.5 to 5.0 moles per mole of the compound represented by the formula (IIa) is exemplified, and preferably, 2 to 4 moles is recommended.

As the organic solvent, toluene, xylene, THF, DMF or a mixed solvent thereof with an alcohol (such as methanol or ethanol) are exemplified.

As the reaction temperature, for example, a temperature of from room temperature to a reflux temperature of the solvent to be used is exemplified, and generally, the reaction is completed in 8 to 72 hours.

Here, as the compound represented by the formula (IIIa), 3-furylboronic acid, 2-furylboronic acid, 3-thienylboronic acid, 2-thienylboronic acid and the like are exemplified.

### Step 2b

The compound represented by the formula (IIa) is reacted with a compound represented by the formula (IIIb) in an organic solvent in the presence of PdCl₂(dppf)₂.CH₂Cl₂ and copper iodide, whereby a compound represented by the formula (IV) is obtained.

As the used amount of the compound represented by the formula (IIIb), an amount of from 1 to 3 moles per mole of the compound represented by the formula (IIa) is exemplified, and preferably, 1.5 moles is recommended. Further, as the used amount of PdCl₂(dPPf)₂.CH₂Cl₂, an amount of from 0.05 to 0.2 moles per mole of the compound represented by the formula (IIa) is exemplified, and preferably, 0.1 moles is recommended. Further, as the used amount of copper iodide, an amount of from 1 to 5 moles per mole of the compound represented by the formula (IIa) is exemplified, and preferably, 4 moles is recommended.

As the organic solvent, THF, DMF and the like are exemplified.

As the reaction temperature, a temperature of from 50 to 100° C. is exemplified, preferably, a temperature of from 60 to 80° C. is recommended, and generally, the reaction is completed in 1 to 6 hours.

Here, as the compound represented by the formula (IIIb), 4-(tributylstannyl)-1,3-thiazole, 2-(tributylstannyl)-1,3-thiazole, 5-(tributylstannyl)-1,3-thiazole and the like are exemplified.

### Step 3

The compound represented by the formula (IV) is hydrolyzed in an organic solvent using a base, whereby a compound represented by the formula (IVa) is obtained.

As the method of hydrolysis, a method known to those skilled in the art can be used. For example, hydrolysis may be carried out in a mixed solvent of a solvent such as methanol or ethanol with water using a base such as sodium hydroxide or potassium hydroxide, preferably potassium hydroxide.

### Step 4

The compound represented by the formula (IVa) and a compound represented by the formula (Va) are subjected to amidation reaction in an organic solvent, whereby the compound represented by the formula (I-1) is obtained.

As to the method of amidation, the amidation can be carried out by a conventionally known method. For example, a method in which the compound represented by the formula (IVa) is reacted with the compound represented by the formula (Va) in the presence of a condensing agent, and a method in which the carboxylic acid moiety of the compound represented by the formula (IVa) is activated by a conventionally known method to form a reactive derivative and then, the derivative and the compound represented by the formula (Va) are amidated are exemplified (see “Pepuchido Gosei no Kiso to Jikken”, Nobuo Izumiya, et al., Maruzen Co., Ltd., 1983 for both methods).

As the reaction using a condensing agent, for example, the following method is exemplified.

That is, the compound represented by the formula (IVa) and the compound represented by the formula (Va) are condensed using a condensing agent in a reaction solvent, whereby the compound represented by the formula (I-1) is obtained.

As the used amount of the compound represented by the formula (Va), an amount of from 1 to 3 moles per mole of the compound represented by the formula (IVa) is exemplified.

As the condensing agent, dicyclohexylcarbodiimide, 1-ethyl-3-(3-dimethylaminopropyl)-carbodiimide, O-(7-azabenzotriazol-1-yl)-1,1,3,3-tetra-methyluronium hexafluorophosphate (hereinafter referred to as “HATU”), and the like are exemplified, and as the used amount thereof, an amount of from 1 to 3 moles per mole of the compound represented by the formula (IVa) is exemplified.

Further, for the purpose of accelerating the reaction, hydroxybenzotriazole (hereinafter referred to as “HOBT”) or the like may be added to the reaction system. As the used amount of HOBT, an amount of from 1 to 3 moles per mole of the compound represented by the formula (IVa) is exemplified.

As the reaction solvent, THF, 1,4-dioxane, N,N-dimethylformamide, DMSO, dichloromethane, chloroform, pyridine or a mixed solvent thereof is exemplified.

As the reaction temperature, a temperature of from 20 to 100° C. is exemplified, preferably, a temperature of from 20 to 50° C. is recommended, and generally, the reaction is completed in 1 to 24 hours.

Here, as the compound represented by the formula (Va), 2-(4-methylpiperazin-1-yl)ethylamine, 2-(1-methylpiperidin-4-yl)ethanamine, 2-methyldecahydro isoquinolin-6-amine and the like are exemplified.

### Step 5

The compound represented by the formula (IIa) is hydrolyzed using a base, whereby a compound represented by the formula (IIb) is obtained. This step can be carried out according to Step 3.

### Step 6

The compound represented by the formula (IIb) and the compound represented by the formula (Va) are subjected to amidation reaction in an organic solvent, whereby a compound represented by the formula (VI) is obtained. This step can be carried out according to Step 4.

### Step 7

The compound represented by the formula (VI) is reacted with the compound represented by the formula (IIIa) or (IIIb) in an organic solvent, whereby the compound represented by the formula (I-1) is obtained. This step can be carried out according to Step 2 (Step 2a or Step 2b).

### Production Method 2

Production Method 2 is a method for producing a compound represented by the formula (I-2).

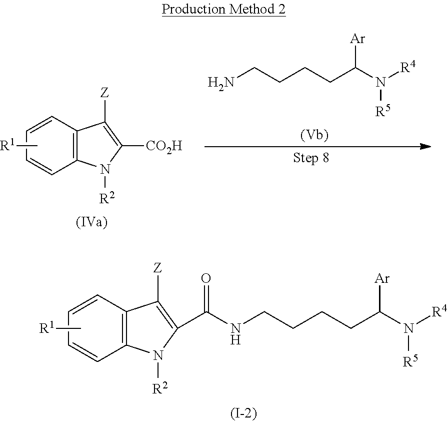

In the formula, each symbol has the same meanings as described above.

### Step 8

The compound represented by the formula (IVa) and a compound represented by the formula (Vb) are subjected to amidation reaction in an organic solvent, whereby the compound represented by the formula (I-2) is obtained. This step can be carried out according to Step 4.

As the compound represented by the formula (Vb), 5-(2-furyl)-5-pyrrolidin-1-ylpentan-1-amine, N-methyl-1-pyridin-2-ylpentan-1,5-diamine and the like are exemplified.

### Production Method 3

Production Method 3 is a method for producing a compound in which Z is H in the compound represented by the formula (I-1).

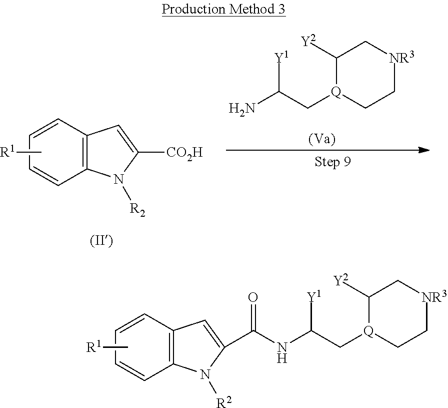

In the formula, each symbol has the same meanings as described above.

### Step 9

A compound represented by the formula (II′) and the compound represented by the formula (Va) are subjected to amidation reaction in an organic solvent, whereby the compound in which Z is H in the compound represented by the formula (I-1) is obtained. This step can be carried out according to Step 4. Incidentally, the compound represented by the formula (II′) can be obtained by hydrolyzing the compound represented by the formula (II) according to Step 3.

In the above-mentioned each production methods, in the case where amino, hydroxy, carboxyl, oxo, carbonyl or the like which is not involved in the reaction is present in the reaction substance, after the amino, hydroxy, carboxyl, oxo or carbonyl is properly protected by a protective group for amino, a protective group for hydroxy, a protective group for carboxyl or a protective group for oxo or carbonyl, each reaction of the above-mentioned production methods is carried out, and the protective group can be removed after the reaction.

The method for introducing and removing the protective group varies depending on the type of the protective group, the stability of the objective compound or the like, however, it can be carried out by, for example, the method described in Protective Groups in Organic Synthesis, T. W. Greene, John Wiley & Sons, (1981) or a modified method thereof, for example, by solvolysis using an acid or a base, i.e., by a method of treating the compound with, for example, 0.01 moles to a large excess amount of an acid, preferably trifluoroacetic acid, formic acid, hydrochloric acid or the like, or with an equimolar amount to a large excess amount of a base, preferably potassium hydroxide, calcium hydroxide or the like; by chemical reduction using a metal hydride complex or the like; or by catalytic reduction using a palladium-carbon catalyst, a Raney nickel catalyst or the like.

The protective group for amino is not particularly limited as long as it has its function, and examples thereof include aralkyl such as benzyl, p-methoxybenzyl and trityl; lower alkanoyl such as acetyl and pivaloyl; benzoyl; lower alkyloxycarbonyl such as methoxycarbonyl, ethoxycarbonyl and tert-butoxycarbonyl; alkyloxycarbonyl such as benzyloxycarbonyl; lower alkylsilyl such as trimethylsilyl and tert-butyldimethylsilyl; tetrahydropyranyl; trimethylsilylethoxymethyl; lower alkylsulfonyl such as methylsulfonyl and ethylsulfonyl; and arylsulfonyl such as benzenesulfonyl and toluenesulfonyl, and particularly preferred is acetyl, benzoyl, tert-butoxycarbonyl, trimethylsilylethoxymethyl, methylsulfonyl or the like.

The protective group for hydroxy is not particularly limited as long as it has its function, and examples thereof include lower alkyl such as methyl, ethyl and tert-butyl; lower alkylsilyl such as trimethylsilyl and tert-butyldimethylsilyl; lower alkyloxymethyl such as methoxymethyl and 2-methoxyethoxymethyl; tetrahydropyranyl; trimethylsilylethoxymethyl; aralkyl such as benzyl, p-methoxybenzyl and 2,3-dimethoxybenzyl; and acyl such as acetyl, and particularly preferred is methyl, methoxymethyl, tetrahydropyranyl, trityl, trimethylsilylethoxymethyl, tert-butyldimethylsilyl, acetyl or the like.

The protective group for carboxyl is not particularly limited as long as it has its function, and examples thereof include lower alkyl such as methyl, ethyl and tert-butyl; halo-lower alkyl such as 2,2,2-trichloroethyl; lower alkenyl such as 2-propenyl; and aralkyl such as benzyl, p-methoxybenzyl, benzhydryl and trityl, and particularly preferred is methyl, ethyl and tert-butyl, 2-propenyl, benzyl, p-methoxybenzyl, benzhydryl or the like.

The protective group for carbonyl is not particularly limited as long as it has its function, and examples thereof include acetals and ketals such as ethylene ketal, dimethyl ketal and S,S′-dimethyl ketal.

The thus obtained compound represented by the formula (I) can be easily isolated and purified by a common separation method such as solvent extraction, recrystallization, column chromatography or preparative thin-layer chromatography.

### Pharmaceutical Composition Containing Compound Represented by Formula (I)

The compound represented by the formula (I) can be administered orally or parenterally, and it can be expected that by formulating the compound into a preparation suitable for such an administration route, the compound has usefulness in the prevention or treatment of cardiovascular diseases such as hypertension, arteriosclerosis, renal diseases, heart diseases and vasospasm; bulimia; and metabolic diseases such as obesity, diabetes, abnormal hormone secretion, hypercholesterolemia, hyperlipidemia, gout and fatty liver. Further, the compound can be used as a preventive or remedy for pain, circadian rhythm disorder, atherosclerosis, obesity-related gastroesophageal reflux, obesity hypoventilation syndrome (Pickwickian syndrome), hypertriglyceridemia, low HDL cholesterolemia, cardiovascular diseases such as coronary heart diseases (CID), peripheral vascular diseases and sudden death, pain, osteoporosis-related diseases, low back pain, anesthetic hypersensitivity and the like, particularly as a preventive or remedy for obesity.

When the compound of the invention is actually used clinically, the compound can be generally administered after it is formulated into any of various preparations with a pharmaceutically acceptable carrier in accordance with the administration mode thereof. As the carrier at this time, any of various carriers commonly used in the pharmaceutical field can be used, and specific examples thereof include gelatin, lactose, sucrose, titanium oxide, starch, crystalline cellulose, hydroxypropyl methyl cellulose, carboxymethyl cellulose, cornstarch, microcrystalline wax, white petrolatum, magnesium aluminometasilicate, anhydrous calcium phosphate, citric acid, trisodium citrate, hydroxypropyl cellulose, sorbitol, sorbitan fatty acid ester, polysorbate, sucrose fatty acid ester, polyoxyethylene, hydrogenated castor oil, polyvinylpyrrolidone, magnesium stearate, light silicic acid anhydride, talc, a vegetable oil, benzyl alcohol, gum Arabic, propylene glycol, polyalkylene glycol, cyclodextrin and hydroxypropyl cyclodextrin.

Examples of the dosage form of the preparation to be formulated using such a carrier include solid preparations such as tablets, capsules, granules, powders and suppositories; and liquid preparations such as syrups, elixirs and injections, and these preparations can be prepared according to a common procedure in the pharmaceutical field. Incidentally, the liquid preparation may be also in the form that the compound and carrier are dissolved or suspended in water or another suitable vehicle before use. Further, particularly in the case of an injection, the compound and carrier may be dissolved or suspended in physiological saline or a glucose solution as needed, and further, a buffer or a preservative may be added thereto.

These preparations can contain the compound of the invention in an amount of from 1 to 99.9% by weight, preferably from 1 to 60% by weight based on the pharmaceutical composition. These preparations may further contain another therapeutically effective compound.

That is, the invention provides a pharmaceutical composition containing a pharmaceutically acceptable carrier and a therapeutically effective amount of the compound of the invention or a pharmaceutically acceptable salt thereof.

Here, the therapeutically effective amount means an amount of a medicament which induces a biological or medical event of tissue, system, animal or human and was found by a researcher, a veterinarian, a physician, or any other clinicians.

That is, the dose and administration frequency in the case where the compound of the invention is used for preventing or treating a disease as described above can be varied depending on the sex, age, body weight, and severity of symptoms of a patient and the type and range of intended treatment effect. However, generally, in the case of oral administration, the administration can be performed at a dose of from 0.001 to 50 mg per kg of body weight per day once or in divided doses. The dose is preferably from about 0.01 to about 25 mg/kg per day, and more preferably from about 0.05 to about 10 mg/kg per day.

The compound of the invention can be used, as a combination therapy, in combination with an agent effective in hypertension, obesity-related hypertension, a hypertension-related disease, heart hypertrophy, left ventricular hypertrophy, a metabolic disease, obesity, an obesity-related disease or the like (hereinafter referred to as a “co-drug”). Such an agent can be administered simultaneously, separately or sequentially in the prevention or treatment of the above-mentioned disease. In the case where the compound of the invention is used simultaneously with one or more co-drugs, they can be formulated into a pharmaceutical composition in the form of a single dose. However, in the combination therapy, a composition containing the compound of the invention and the co-drug may be administered simultaneously, separately or sequentially to a subject to be treated as different packages. These may also be administered at different times.

The dose of such a co-drug may be based on the dose used in clinical practice, and can be suitably selected depending on the subject to be administered, administration route, disease, combination or the like. The administration mode of the co-drug is not particularly limited, and it is sufficient that the co-drug is combined with the compound of the invention in their clinical administration.

The administration mode includes, for example, 1) single administration of a single preparation containing both a compound of the invention and a co-drug; 2) simultaneous administration of two preparations through the same administration route, in which the two preparations are prepared separately and each separately contain a compound of the invention and a co-drug; 3) separate administration at different times of two preparations through the same administration route, in which the two preparations are prepared separately and each separately contain a compound of the invention and a co-drug; 4) simultaneous administration of two preparations through different administration routes, in which the two preparations are prepared separately and each separately contain a compound of the invention and a co-drug; 5) separate administration at different times of two preparations through different administration routes, in which the two preparations are prepared separately and each separately contain a compound of the invention and a co-drug (for example, administration of a compound of the invention followed by a co-drug, or administration in the reversed order). The dose ratio of the compound of the invention and the co-drug may be suitably determined depending on the subject to which they are administered, the administration route, the disorder of the subject, etc.

The co-drug for use in the invention includes, for example, remedies for diabetes, remedies for hyperlipidemia, remedies for hypertension, remedies for obesity, etc. Two or more different types of such co-drug may be combined in any desired ratio.

The usefulness of the compounds of the invention as medicines is proved, for example, by the following Pharmacological Test Example 1.

### PHARMACOLOGICAL TEST EXAMPLE 1

### QRFP43-Binding Inhibitory Test

A cDNA sequence [Accession No. NM_{—}198179] coding for a human QRFP receptor (GPR103) was cloned in an expression vector pEF1V5-HisB (by Invitrogen). The resulting expression vector was transfected in a host cell CHO-K1 NFAT (3-Lactamase (by Aurora), according to a cationic lipid process [see Proceedings of the National Academy of Sciences of the United States of America, Vol. 84, p. 7413, 1987], thereby producing a QRFP receptor (GPR103).

A membrane sample prepared from the cells having expressed a QRFP receptor (GPR103) was incubated in an assay buffer (50 mM Tris-HCl, 1 mM EDTA and 0.1% BSA, pH 7.4) along with a test compound and 20,000 cpm [¹²⁵I] QRFP43 (by Perkin Elmer) therein, at 25° C. for 1 hour, and then filtered through a glass filter GF/C. After washed with 50 mM Tris-HCl (containing 2 mM EDTA, 10 mM MgCl₂ and 0.04% Tween-20) buffer (pH 7.4), the radioactivity on the glass filter was determined. The non-specific binding was determined in the presence of 1 μM peptide QRFP43, and the 50% inhibitory concentration (IC₅₀) of the test compound to the specific [¹²⁵I] QRFP43 binding was computed [see Endocrinology, Vol. 131, p. 2090, 1992]. The results are shown in Table 1.

TABLE 1

| Example | Chemical structure | IC50 (nM) |
|---|---|---|
| 1 | 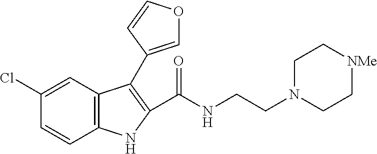 | 2.26 |
| 2 | 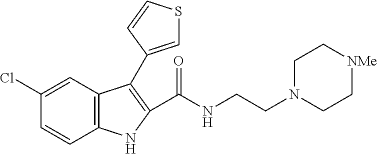 | 4.71 |
| 3 | 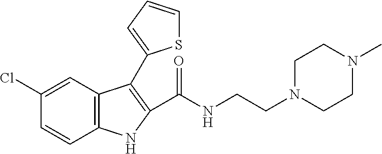 | 4.68 |
| 4 | 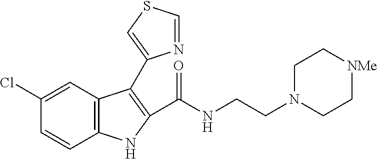 | 14.22 |
| 5 | 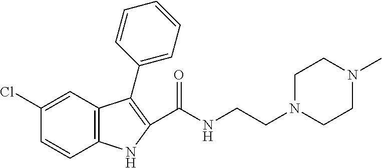 | 11.44 |
| 6 | 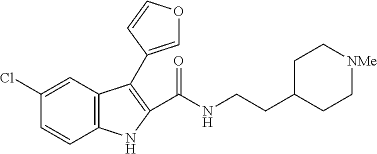 | 6.24 |
| 7 | 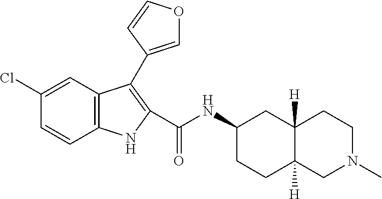 | 9.82 |
| 8 | 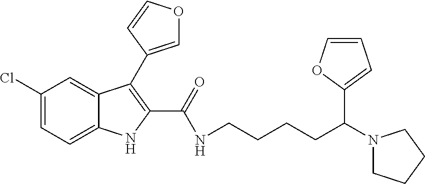 | 0.58 |
| 9 | 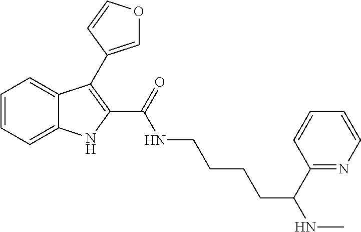 | 1.06 |
| Reference example 1 | 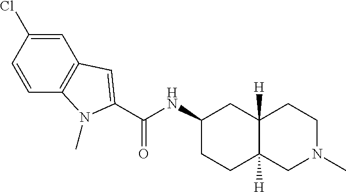 | 22.56 |

As shown in the above table, the compounds of the invention strongly inhibited the binding of [¹²⁵I]QRFP43 to the QRFP43 receptor (GPR103).

From the above results, it can be expected that the compounds according to the invention have usefulness in the prevention or treatment of various diseases related to QRFP43 or 26RFa, for example, cardiovascular diseases such as hypertension, arteriosclerosis, renal diseases, heart diseases and vasospasm; bulimia; and metabolic diseases such as obesity, diabetes, abnormal hormone secretion, hypercholesterolemia, hyperlipidemia, gout and fatty liver. Further the compounds can be used as a preventive or remedy for pain, circadian rhythm disorder, atherosclerosis, obesity-related gastroesophageal reflux, obesity hypoventilation syndrome (Pickwickian syndrome), hypertriglyceridemia, low HDL cholesterolemia, cardiovascular diseases such as coronary heart diseases (CHD), peripheral vascular diseases and sudden death, pain, osteoporosis-related diseases, low back pain, anesthetic hypersensitivity and the like, and particularly as a preventive or remedy for obesity.

### EXAMPLES

Hereinafter, the invention will be more specifically described with reference to Examples, however, the invention is not limited only to these Examples. Incidentally, as a column silica gel, Wakogel TM C-200 (Wako Pure Chemical Industries, Ltd.) was used; as a packed silica gel column, a disposable column (Si series, NH series) (Moritex Corporation), FLASH+™ Cartridge, KP-Sil or FPNH, FLASH 12+M, FLASH 25+S, FLASH 25+M, FLASH 40+M or the like (Biotage Japan), TC-C18 (Agilent Technologies) or Extend-C18 (ZORBAX) was used; and as a fractionation thin-layer chromatograph, Kieselgel 60F254 (Merck) was used. Further, mass spectra were measured using Quattro II (manufactured by Micromass). ¹H-NMR was measured using JNM-AL 400 (manufactured by JEOL) or MERCURYvx 400 (manufactured by Varian, Inc.) and ^{UNITY}INOVA 400 (manufactured by Varian, Inc.); and mass spectra were measured using ZQ 2000 (manufactured by Waters, Inc.).

### Production Example 1-1

### ethyl 3-bromo-5-chloro-1H-indole-2-carboxylate

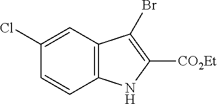

To a DMF solution (500 mL) of commercially available ethyl 5-chloro-1H-indole-2-carboxylate (27 g), NBS (25.8 g) was added, and the resulting solution was stirred overnight at room temperature. To the reaction solution, water (1.5 L) was added and a precipitate was collected by filtration. After the precipitate was dissolved in ethyl acetate (1.3 L), the organic layer was washed with water and then saturated brine. The organic layer was dried over anhydrous magnesium sulfate, and the solvent was distilled off under reduced pressure. To the residue, a chloroform-methanol mixed solvent (10:1, 50 mL) was added to suspend the residue, and the suspension was filtered. The residue was washed with chloroform (12 mL×3) and dried under reduced pressure, whereby the title compound (29.1 g) was obtained as a pale yellow solid.

### Production Example 1-2

### ethyl 5-chloro-3-(3-furyl)-1H-indole-2-carboxylate

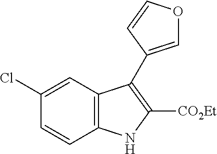

A toluene-ethanol mixed solvent (1:1, 600 mL) containing the compound obtained in Production example 1-1 (14 g), 3-furylboronic acid (7.8 g), tetrakis(triphenyl phosphine)palladium(0) (10.7 g) and a 1 M aqueous sodium carbonate solution (93 mL) was heated under reflux overnight at 100° C. under a nitrogen atmosphere. The reaction solution was concentrated under reduced pressure, and to the residue, chloroform was added, and the chloroform layer was washed with a saturated aqueous sodium hydrogen carbonate solution and then saturated brine. The chloroform layer was dried over anhydrous magnesium sulfate, and the solvent was distilled off under reduced pressure. To the residue, chloroform (50 mL) was added to suspend the residue, and the suspension was filtered. The residue was washed with chloroform (25 mL×4) and dried under reduced pressure, whereby the title compound (12.4 g) was obtained as a white solid.

### Production Example 1-3

### 5-chloro-3-(3-furyl)-1H-indole-2-carboxylic acid

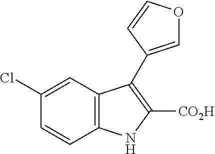

To an ethanol-water mixed solvent (14:1, 375 mL) of the compound obtained in Production Example 1-2 (13.5 g), potassium hydroxide (7.9 g) was added, and the resulting solution was stirred at 80° C. for 3 hours. The reaction solution was concentrated under reduced pressure, water (150 mL) was added thereto, and then, the mixture was acidified with 5 N hydrochloric acid (30 mL). A precipitate was collected by filtration and washed with water (270 mL) and then dried under reduced pressure, whereby the title compound (12 g) was obtained as a gray-brown solid.

### Production Example 2-1

### ethyl 5-chloro-3-(3-thienyl)-1H-indole-2-carboxylate

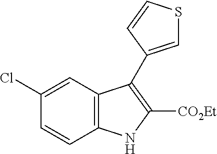

The title compound (7.9 g) was obtained as a gray solid by the same procedure as in Production example 1-2 using the compound obtained in Production example 1-1 (15 g), 3-thienylboronic acid (9.5 g), tetrakis(triphenylphosphine) palladium(0) (11.5 g) and a 1 M aqueous sodium carbonate solution (99 mL).

### Production Example 2-2

### 5-chloro-3-(3-thienyl)-1H-indole-2-carboxylic acid

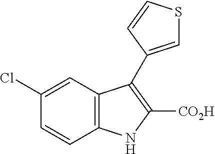

The title compound (7.1 g) was obtained as a gray solid by the same procedure as in Production example 1-3 using the compound obtained in Production example 2-1 (7.9 g) and potassium hydroxide (4.4 g).

### Production Example 3-1

### 3-bromo-5-chloro-1H-indole-2-carboxylic acid

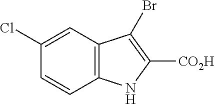

The title compound (7.2 g) was obtained as a white solid by the same procedure as in Production example 1-3 using the compound obtained in Production example 1-1 (8 g) and potassium hydroxide (3.0 g).

### Production Example 3-2

### 3-bromo-5-chloro-N-[2-(4-methylpiperazin-1-yl)ethyl]-1H-indole-2-carboxamide

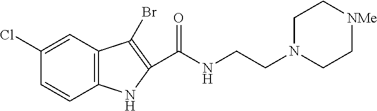

To a DMF solution (70 mL) containing the compound obtained in Production example 3-1 (2.6 g), 2-(4-methyl piperazin-1-yl)ethylamine (1.5 g) and HATU (4.0 g), diisopropylethylamine (5.0 mL) was added, and the resulting solution was stirred overnight at room temperature. Water was added to the reaction solution and the solution was extracted with chloroform. The chloroform layer was dried over anhydrous magnesium sulfate, and the solvent was distilled off under reduced pressure. To the residue, chloroform was added to suspend the residue, and the suspension was filtered. The residue was washed with chloroform and dried under reduced pressure, whereby the title compound (2.2 g) was obtained as a white solid.

### Production Example 4-1

### ethyl 5-chloro-3-(1,3-thiazol-4-yl)-1H-indole-2-carboxylate

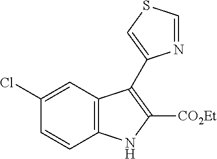

A THF solution (20 mL) containing the compound obtained in Production example 1-1 (1.2 g), 4-(tributylstannyl)1,3-thiazole (2.2 g), [1,1′-bis(diphenylphosphino)ferrocene-palladium(II) dichloride dichloromethane complex (324 mg) and copper iodide(I) (3.0 g) was heated under reflux under a nitrogen atmosphere for 6 hours. The reaction solution was filtered using Celite and the filtrate was concentrated under reduced pressure. The residue was purified by silica gel column chromatography (hexane:ethyl acetate=1:1), whereby the title compound (340 mg) was obtained as a yellow solid.

### Production Example 4-2

### 5-chloro-3-(1,3-thiazol-4-yl)-1H-indole-2-carboxylic acid

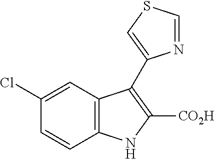

The title compound (300 mg) was obtained as a yellow-brown solid by the same procedure as in Production example 1-3 using the compound obtained in Production example 4-1 (340 mg) and potassium hydroxide (240 mg).

### Production Example 5-1

### tert-butyl 6-oxo-3,4,6,7,8,8a-hexahydroisoquinoline-2 (1H)-carboxylate

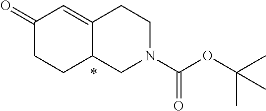

To a toluene solution (500 mL) of commercially available tert-butyl 4-oxopiperidine-1-carboxylate (60.0 g), pyrrolidine (42.8 g) and p-toluenesulfonic acid monohydrate (610 mg) were added, and the resulting solution was heated under reflux for 3 days using a Dean-Stark apparatus. The reaction solution was concentrated under reduced pressure. To a toluene solution (400 mL) of the resulting residue, a toluene solution (20 mL) of methyl vinyl ketone (27.6 mL) was added dropwise at a temperature not higher than 30° C. over 15 minutes. After completion of the dropwise addition, the resulting solution was stirred at room temperature for 3 hours and then at 80° C. for 2.5 hours. After the reaction solution was cooled to room temperature, an aqueous solution (100 mL) containing acetic acid (30 mL) and potassium acetate (10 g) was added thereto, and the resulting solution was stirred at the same temperature for 30 minutes. The organic layer was separated and washed with water and then saturated brine. Then, the organic layer was dried over anhydrous sodium sulfate, and the solvent was distilled off under reduced pressure. The resulting residue was purified by silica gel column chromatography (hexane:ethyl acetate=5:1 to 3.5:1), and to the resulting solid, a hexane-diethyl ether mixed solvent was added to suspend the solid. The suspension was filtered, and the residue was dried under reduced pressure, whereby the title compound in racemic form (18.2 g) was obtained as a white solid. The compound in racemic form (18 g) was resolved using an optically active column (column: manufactured by Daicel Chemical Industries, Ltd., Chiralpack AD, mobile phase: hexane-ethanol (75:25)), and a fraction with a faster retention time (8.6 g) and a fraction with a later retention time (7.6 g) were collected as optically active samples, respectively. In the subsequent reaction, the optically active sample with a longer retention time was used.

### Production Example 5-2

### tert-butyl 6-hydroxyoctahydroisoquinoline-2(1H)-carboxylate

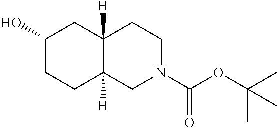

An ammonia solution (200 mL) was cooled to −78° C., and lithium (1.0 g) was added thereto little by little. After it was confirmed that lithium was dissolved therein, a THF solution (80 mL) of the compound obtained in Production example 5-1 (5.4 g) was added dropwise thereto at the same temperature over 20 minutes. After completion of the dropwise addition, the resulting solution was stirred at −78° C. for 30 minutes, and then, t-butanol (2.25 mL) was added dropwise thereto at the same temperature. After completion of the dropwise addition, the resulting solution was stirred at −78° C. for 30 minutes, and to the reaction solution, a saturated aqueous ammonium chloride solution was added. THF was distilled off under reduced pressure, followed by filtration. The residue was washed with ethyl acetate. The filtrate was concentrated under reduced pressure, and to a THF solution (50 mL) of the resulting residue, pentafluorophenol (2.2 mL) was added, and the resulting solution was cooled to 0° C. To this solution, sodium borohydride (810 mg) was added, and the resulting solution was stirred at the same temperature for 1 hour. To the reaction solution, a 1 N aqueous sodium hydroxide solution was added and the resulting solution was extracted with ethyl acetate. The ethyl acetate layer was washed with saturated brine and then dried over anhydrous sodium sulfate. The solvent was distilled off under reduced pressure, and the resulting residue was purified by silica gel column chromatography (hexane:ethyl acetate=3.5:1 to 2.5:1), whereby the title compound (5.0 g) in the form of a colorless oily substance was obtained as a single optically active isomer (the arrangement in the structural formula is a relative configuration).

### Production Example 5-3

### tert-butyl 6-azidooctahydroisoquinoline-2(1H)-carboxylate

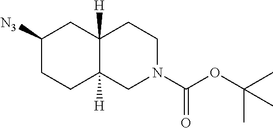

A THF solution (50 mL) of the compound obtained in Production example 5-2 (2.9 g) was cooled to 0° C., and triethylamine (1.1 g) and subsequently, methanesulfonyl chloride (0.97 mL) were added thereto, and the resulting solution was stirred at the same temperature for 3 hours. Water was added to the reaction solution, and the resulting solution was extracted with ethyl acetate. The organic layer was washed with water and then saturated brine. After the organic layer was dried over anhydrous sodium sulfate, the solvent was distilled off under reduced pressure. The resulting residue was dissolved in DMF (25 mL) and sodium azide (1.5 g) was added thereto, and the resulting solution was stirred at 80° C. for 20 hours. After the reaction solution was cooled to room temperature, water was added thereto, and the solution was extracted with ethyl acetate. The organic layer was washed with water and then saturated brine. After the organic layer was dried over anhydrous sodium sulfate, the solvent was distilled off under reduced pressure. The resulting residue was purified by silica gel column chromatography (hexane:ethyl acetate=30:1 to 25:1), whereby the title compound was obtained as a colorless oily substance. The same procedure was carried out using the compound obtained in Production example 5-2 (2.1 g) and the resulting product was combined with the previously obtained colorless oily substance, whereby the title compound (3.3 g) was obtained as a single optically active isomer (the arrangement in the structural formula is a relative configuration).

### Production Example 5-4

### tert-butyl 6-{[5-chloro-1-methyl-1H-indol-2-yl]carbonyl}amino)octahydroisoquinoline-2(1H)-carboxylate

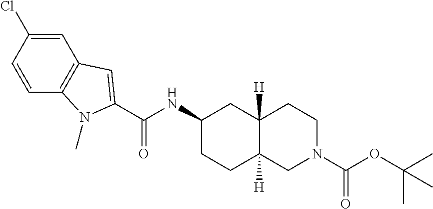

To a methanol solution (100 mL) of the compound obtained in Production example 5-3 (3.3 g), 10% palladium carbon (500 mg) was added, and the resulting solution was stirred overnight at room temperature under 1 atm hydrogen atmosphere. The reaction solution was filtered and the filtrate was concentrated under reduced pressure. To a DMF (70 mL) solution of the residue, commercially available 5-chloro-1-methyl-1H-indole-2-carboxylic acid (2.4 g), HATU (4.4 g) and subsequently diisopropylethylamine (4.1 mL) were added, and the resulting solution was stirred overnight at room temperature. Water was added to the reaction solution, and the solution was extracted with ethyl acetate. The organic layer was dried over anhydrous sodium sulfate, and the solvent was distilled off under reduced pressure. The resulting residue was purified by silica gel column chromatography (hexane:ethyl acetate=4:1 to 1:2), whereby the title compound (3.2 g) was obtained as a single optically active isomer (the arrangement in the structural formula is a relative configuration).

### Production Example 6-1

### 6-azido-2-methyldecahydroisoquinoline

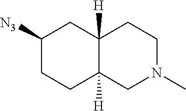

The compound obtained in Production example 5-3 (150 mg) was dissolved in trifluoroacetic acid (5 mL), and the resulting solution was stirred at room temperature for 10 minutes. After the reaction solution was alkalinized by adding a 5 N sodium hydroxide solution thereto, the alkalinized solution was extracted with chloroform. The chloroform layer was dried over anhydrous sodium sulfate and concentrated under reduced pressure, whereby a crude product of 6-azidodecahydroisoquinoline was obtained. To a 1% acetic acid-chloroform solution (7 mL) of the resulting crude product, formalin (43.8 μL) and sodium triacetoxy borohydride (125 mg) were added, and the resulting solution was stirred at room temperature for 1 hour. To the reaction solution, a 1 N sodium hydroxide solution was added, and the solution was extracted with chloroform. The chloroform layer was dried over anhydrous sodium sulfate and then concentrated under reduced pressure, whereby a crude product (104 mg) of the title compound was obtained as a single optically active isomer (the arrangement in the structural formula is a relative configuration). This product was used in the subsequent reaction without purification.

### Production Example 7-1

### tert-butyl [5-(2-furyl)-5-oxopentyl]carbamate

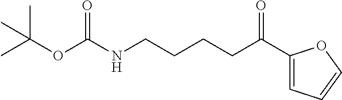

To a THF solution (10 mL) of furan (188 mg), an n-hexane solution (1.7 mL) of 1.61 M n-butyl lithium was added dropwise at −78° C., and the resulting solution was stirred at the same temperature for 40 minutes. To the reaction solution, a THF solution (0.5 mL) of commercially available tert-butyl 2-oxopiperidine-1-carboxylate (500 mg) was added at −78° C., and after the temperature of the solution was raised to −20° C., the solution was stirred at the same temperature for 2 hours. To the reaction solution, a saturated aqueous ammonium chloride solution was added, and the solution was extracted with ethyl acetate. The organic layer was dried over anhydrous magnesium sulfate and the solvent was distilled off under reduced pressure. The resulting residue was purified by silica gel column chromatography (hexane:ethyl acetate=3:2), whereby the title compound (254 mg) was obtained as a colorless oily substance.

### Production Example 7-2

### tert-butyl [5-(2-furyl)-5-pyrrolidin-1-ylpentyl]carbamate

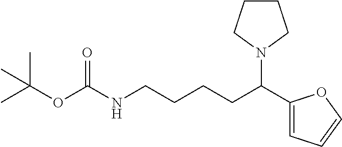

To a THF solution (2 mL) containing the compound obtained in Production example 7-1 (100 mg) and pyrrolidine (0.12 mL), titanium tetraisopropoxide (0.22 mL) was added, and the resulting solution was stirred at 60° C. for 6 hours. After the reaction solution was cooled to 0° C., ethanol (2 mL) and then sodium borohydride (56.6 mg) were added, and the solution was stirred overnight at room temperature. Water was added to the reaction solution, and the solution was extracted with chloroform. The chloroform layer was dried over anhydrous magnesium sulfate and the solvent was distilled off under reduced pressure. The resulting residue was purified by silica gel column chromatography (hexane:ethyl acetate=1:4), whereby the title compound (47 mg) was obtained.

### Production Example 7-3

### 5-(2-furyl)-5-pyrrolidin-1-ylpentan-1-amine hydrochloride

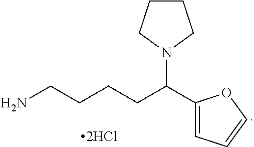

To a methanol solution (1 mL) of the compound obtained in Production example 7-2 (47 mg), a 4 N hydrochloric acid-dioxane solution (0.5 mL) was added, and the resulting solution was stirred overnight at room temperature. The reaction solution was concentrated under reduced pressure, whereby the title compound (38.4 mg) was obtained. This compound was used in the subsequent reaction without purification.

### Production Example 8-1

### tert-butyl (5-oxo-5-pyridin-2-ylpentyl)carbamate

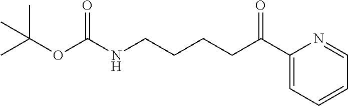

To a THF solution (15 mL) containing commercially available 2-bromopyridine (1.4 g) and N,N,N′-N′-tetramethyl ethylene diamine (1.1 mL), a hexane solution (4.6 mL) of 1.61 M n-butyl lithium was added dropwise at −78° C., and the resulting solution was stirred at the same temperature for 1.5 hours. To the reaction solution, a THF solution (2 mL) of commercially available tert-butyl 2-oxopiperidine-1-carboxylate (990 mg) was added at −78° C., and the resulting solution was stirred at the same temperature for 2 hours. To the reaction solution, a saturated aqueous ammonium chloride solution was added, and the solution was extracted with chloroform. The chloroform layer was washed with saturated brine and dried over anhydrous magnesium sulfate, and then the solvent was distilled off under reduced pressure. The resulting residue was purified by silica gel column chromatography (hexane:ethyl acetate=3:2), whereby the title compound (747 mg) was obtained.

### Production Example 8-2

### tert-butyl [5-(methylamino)-5-pyridin-2-ylpentyl]carbamate

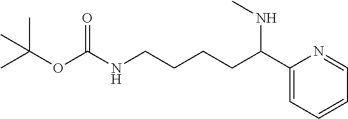

To a THF solution (2.0 mL) containing the compound obtained in Production example 8-1 (906 mg) and 10 M methylamine, titanium tetraisopropoxide (1.9 g) was added, and the resulting solution was stirred at room temperature for 2.5 hours. After the reaction solution was cooled to 0° C., ethanol (5.4 mL) and then sodium borohydride (493 mg) were added, and the solution was stirred at room temperature for 1 hour. To the reaction solution, water and a saturated aqueous sodium hydrogen carbonate solution were added, and the solution was extracted with chloroform. The chloroform layer was washed with saturated brine and dried over anhydrous magnesium sulfate, and then, the solvent was distilled off under reduced pressure. The resulting residue was purified by NH-silica gel column chromatography (ethyl acetate), whereby the title compound (688 mg) was obtained.

### Production Example 8-3

### N-methyl-1-pyridin-2-ylpentan-1,5-diamine hydrochloride

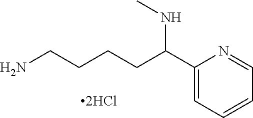

The title compound (630 mg) was obtained by the same procedure as in Production example 7-3 using the compound obtained in Production example 8-2 (728 mg) and a 4 N hydrochloric acid-dioxane solution (2.5 mL).

### Production Example 9-1

### ethyl 3-(3-furyl)-1H-indole-2-carboxylate

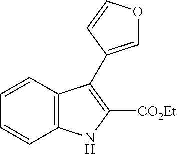

A toluene-ethanol mixed solvent (1:1, 60 mL) containing commercially available ethyl 3-bromo-1H-indole-2-carboxylate (1.5 g), 3-furylboronic acid (939 mg), tetrakis(triphenylphosphine)palladium(0) (1.29 g) and a 1 M aqueous sodium carbonate solution (11.2 mL) was heated under reflux overnight at 100° C. under a nitrogen atmosphere. The reaction solution was concentrated under reduced pressure, and to the residue, water was added and the solution was extracted with chloroform. The chloroform layer was dried over anhydrous magnesium sulfate, and the solvent was distilled off under reduced pressure. The resulting residue was purified by silica gel column chromatography (hexane:ethyl acetate=3:1), whereby the title compound (340 mg) was obtained as a pale yellow solid.

### Production Example 9-2

### 3-(3-furyl)-1H-indole-2-carboxylic acid

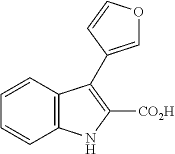

The title compound (300 mg) was obtained as a gray solid by the same procedure as in Production example 1-3 using the compound obtained in Production example 9-1 (340 mg) and potassium hydroxide (224 mg).

### Example 1

### 5-chloro-3-(3-fury)-N-[2-(4-methylpiperazin-1-yl)ethyl]-1H-indole-2-carboxamide

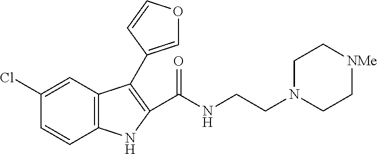

A DMF solution (340 mL) containing the compound obtained in Production example 1-3 (11.6 g), 2-(4-methylpiperazin-1-yl)ethylamine (7.0 g) and HATU (18.5 g) was ice-cooled to 15° C., and diisopropylethylamine (23.2 mL) was added thereto and the resulting solution was stirred overnight at room temperature. The reaction solution was poured into water (1.5 L), and the mixture was stirred at room temperature for 5 minutes. A precipitate was collected by filtration and washed with water and then dissolved in chloroform (600 mL). The chloroform layer was washed with a saturated aqueous sodium hydrogen carbonate solution and then dried over anhydrous magnesium sulfate. The solvent was distilled off under reduced pressure, and the resulting residue was purified by NH-silica gel column chromatography (hexane:ethyl acetate=2:1 to 1:3), whereby the title compound (8.8 g) was obtained as a pale yellow solid.

¹H-NMR (400 MHz, CDCl₃, δppm): 2.30 (3H, s), 2.32-2.50 (8H, m), 2.47 (2H, t, J=5.8 Hz), 3.51 (2H, dt, J=5.8 Hz, 5.8 Hz), 6.63 (1H, dd, J=1.9, 0.9 Hz), 7.03 (1H, br s), 7.25 (1H, m), 7.39 (1H, dd, J=8.8 Hz, 0.6 Hz), 7.49 (1H, m), 7.66 (1H, m), 7.70 (1H, m), 9.78 (1H, s).

ESI-MS Found: m/z 387[M+H]⁺

### Example 2

### 5-chloro-N-[2-(4-methylpiperazin-1-yl)ethyl]-3-(3-thienyl)-1H-indole-2-carboxamide

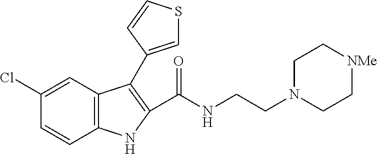

A crude product of the title compound was obtained by the same procedure as in Example 1 using the compound obtained in Production example 2-2 (6 g), 2-(4-methyl piperazin-1-yl)ethylamine (3.4 g), HATU (9.0 g) and diisopropylethylamine (11.3 mL). This crude product was purified by NH-silica gel column chromatography (hexane:ethyl acetate=1:1 to 1:4), whereby the title compound (3.3 g) was obtained as a pale yellow solid.

¹H-NMR (400 MHz, CDCl₃, δppm): 2.28 (3H, s), 2.30-2.45 (8H, m), 2.42 (2H, t, J=5.8 Hz), 3.48 (2H, dt, J=5.8, 5.8 Hz), 6.75 (1H, br s), 7.26 (2H, m), 7.39 (1H, d, J=8.6 Hz), 7.48 (2H, m), 7.55 (1H, m), 9.80 (1H, s).

ESI-MS Found: m/z 403[M+H]⁺

### Example 3

### 5-chloro-N-[2-(4-methylpiperazin-1-yl)ethyl]-3-(2-thienyl)-1H-indole-2-carboxamide

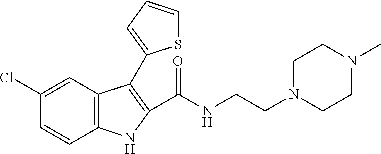

A crude product of the title compound was obtained by the same procedure as in Production example 1-2 using the compound obtained in Production example 3-2 (50 mg), 2-thienylboronic acid (25 mg), tetrakis(triphenyl phosphine)palladium(0) (30 mg) and a 1 M aqueous sodium carbonate solution (0.26 mL). This crude product was purified by NH-silica gel column chromatography (chloroform:methanol=50:1), whereby the title compound (13.9 mg) was obtained as a pale yellow oily substance.

¹H-NMR (400 MHz, CDCl₃, δppm): 2.29 (3H, s), 2.33-2.41 (8H, m), 2.43 (2H, t, J=5.8 Hz), 3.49 (2H, dt, J=5.8, 5.8 Hz), 6.92 (1H, br s), 7.21-7.27 (3H, m), 7.41 (1H, d, J=9.0 Hz), 7.53 (2H, m), 10.1 (1H, s)

ESI-MS Found: m/z 403[M+H]⁺

### Example 4

### 5-chloro-N-[2-(4-methylpiperazin-1-yl)ethyl]-3-(1,3-thiazol-4-yl)-1H-indole-2-carboxamide

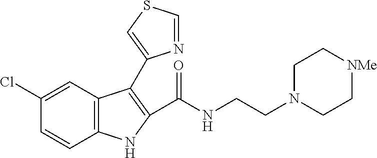

A crude product of the title compound was obtained by the same procedure as in Example 1 using the compound obtained in Production example 4-2 (200 mg), 2-(4-methyl piperazin-1-yl)ethylamine (113 mg), HATU (300 mg) and diisopropylethylamine (0.63 mL). This crude product was purified by NH-silica gel column chromatography (chloroform:methanol=20:1), whereby the title compound (100 mg) was obtained as a colorless oily substance.

¹H-NMR (400 MHz, CDCl₃, δppm): 2.37 (3H, s), 2.49-2.68 (10H, m), 3.64 (2H, dt, J=6.0, 6.0 Hz), 7.30 (1H, dd, J=8.6, 2.0 Hz), 7.42 (1H, d, J=9.0 Hz), 7.71 (1H, d, J=2.0 Hz), 7.86 (1H, d, J=2.0 Hz), 9.03 (1H, d, J=2.0 Hz), 9.76 (1H, s), 10.59 (1H, s).

ESI-MS Found: m/z 404[M+H]⁺

### Example 5

### 5-chloro-N-[2-(4-methylpiperazin-1-yl)ethyl]-3-phenyl-1H-indole-2-carboxamide

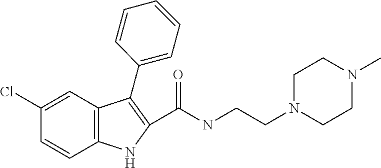

To a DMF solution (1 mL) containing commercially available 5-chloro-3-phenyl-1H-indole-2-carboxylic acid (100 mg), 2-(4-methylpiperazin-1-yl)ethylamine (52 mg) and HATU (141 mg), diisopropylethylamine (0.2 mL) was added and the resulting solution was stirred overnight at room temperature. Water was added to the reaction solution and the solution was extracted with chloroform. After the chloroform layer was dried over anhydrous magnesium sulfate, the solvent was distilled off under reduced pressure, and the resulting residue was purified by NH-silica gel column chromatography (hexane:ethyl acetate=1:1), whereby the title compound (100 mg) was obtained as a yellow solid.

¹H-NMR (400 MHz, CDCl₃, δppm): 2.22-2.32 (8H, m), 2.23 (3H, s), 2.36 (2H, t, J=5.8 Hz), 3.44 (2H, dt, J=5.8, 5.8 Hz), 6.52 (1H, br s), 7.25 (1H, m), 7.41 (2H, m), 7.51 (5H, m), 9.89 (1H, s).

ESI-MS Found: m/z 397[M+H]⁺

### Example 6

### 5-chloro-3-(3-fury)-N-[2-(1-methylpiperidin-4-yl)ethyl]-1H-indole-2-carboxamide

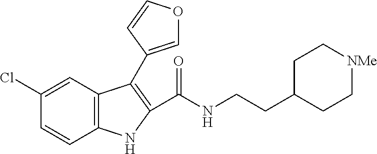

The title compound (106 mg) was obtained as a yellow solid by the same procedure as in Example 5 using the compound obtained in Production example 1-3 (100 mg), 2-(1-methylpiperidin-4-yl) ethanamine (59.8 mg) synthesized according to WO 1996/38420, HATU (160 mg) and diisopropylethylamine (0.2 mL).

¹H-NMR (400 MHz, CDCl₃, δppm): 1.30 (3H, m), 1.44 (2H, m), 1.67 (2H, m), 1.95 (2H, m), 2.31 (3H, s), 2.90 (2H, m), 3.43 (2H, m), 6.36 (1H, m), 6.59 (1H, m) 7.26 (1H, m), 7.39 (1H, d, J=9.0 Hz), 7.46 (1H, d, J=2.0 Hz), 7.67 (1H, m), 7.69 (1H, m), 9.75 (1H, s).

ESI-MS Found: m/z 386[M+H]⁺

### Example 7

### 5-chloro-3-(3-fury)-N-(2-methyldecahydroisoquinolin-6-yl)-1H-indole-2-carboxamide

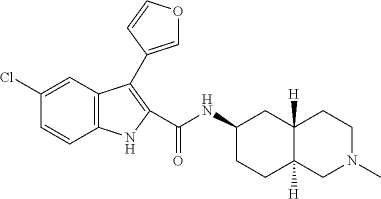

To a methanol solution (20 mL) of the compound obtained in Production example 6-1 (104 mg), 10% palladium carbon (30 mg) was added, and the resulting solution was stirred at room temperature under 1 atm hydrogen atmosphere for 3 hours. The reaction solution was filtered and the filtrate was concentrated under reduced pressure. To a DMF (4 mL) solution of the residue, the compound obtained in Production example 1-3 (126 mg), HATU (183 mg) and subsequently diisopropylethylamine (0.17 mL) were added, and the resulting solution was stirred overnight at room temperature. The reaction solution was concentrated under reduced pressure, and the resulting residue was purified by NH-silica gel column chromatography (chloroform:methanol=30:1), whereby the title compound (122 mg) in the form of a yellow solid was obtained as a single optically active isomer (the arrangement in the structural formula is a relative configuration).

¹H-NMR (400 MHz, CDCl₃, δppm): 0.51 (1H, m), 0.67 (1H, m), 1.27 (3H, m), 1.55-1.72 (2H, m) 1.79 (1H, m), 1.88 (1H, m), 2.28 (3H, s), 2.71 (1H, m), 2.88 (1H, m), 4.40 (1H, m), 6.62 (1H, m), 6.68 (1H, m), 7.27 (1H, m), 7.40 (2H, m), 7.69 (1H, m), 7.72 (1H, m), 9.67 (1H, s).

ESI-MS Found: m/z 412[M+H]⁺

### Example 8

### 5-chloro-3-(3-fury)-N-[5-(2-furyl)-5-pyrrolidin-1-ylpentyl]-1H-indole-2-carboxamide

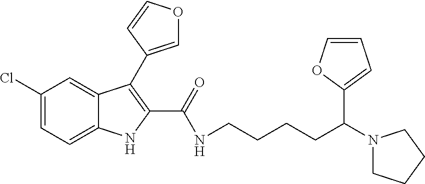

To a DMF solution (1 mL) containing the compound obtained in Production example 7-3 (38.4 mg), the compound obtained in Production example 1-3 (34 mg) and HATU (54.3 mg), diisopropylethylamine (0.091 mL) was added and the resulting solution was stirred overnight. Water was added to the reaction solution and the solution was extracted with chloroform. After the chloroform layer was dried over anhydrous magnesium sulfate, the solvent was distilled off under reduced pressure, and the resulting residue was purified by NH-silica gel column chromatography (chloroform:methanol=96:4), whereby the title compound in racemic form (23.2 mg) was obtained.

¹H-NMR (400 MHz, CDCl₃, δppm): 1.09-1.23 (2H, m), 1.39-1.54 (2H, m) 1.75 (4H, m), 1.89 (2H, m) 2.47 (2H, m), 2.62 (2H, m), 3.34 (2H, m), 3.44 (1H, m), 6.16 (1H, m), 6.29 (1H, m), 6.35 (1H, m), 6.53 (1H, m), 7.25 (1H, m), 7.37 (2H, m), 7.46 (1H, m), 7.64 (2H, m), 9.77 (1H, s).

ESI-MS Found: m/z 466[M+H]⁺

### Example 9

### 3-(3-fury)-N-[5-(methylamino)-5-pyridin-2-ylpentyl]-1H-indole-2-carboxamide

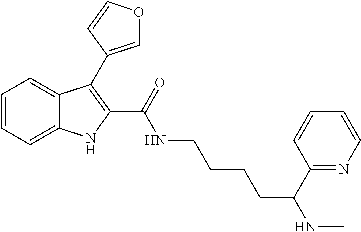

The title compound in racemic form (128.4 mg) was obtained by the same procedure as in Example 8 using the compound obtained in Production example 8-3 (630 mg), the compound obtained in Production example 9-2 (538 mg), HATU (1.4 g) and diisopropylethylamine (2.9 mL). The compound in racemic form (36.2 mg) was resolved using an optically active column (column: manufactured by Daicel Chemical Industries, Ltd., Chiralpack AD-H, mobile phase: hexane-isopropanol (supplemented with 0.1% diethylamine) (1:1), and a fraction with a faster retention time (8.6 mg) and a fraction with a later retention time (10.7 mg) were collected as optically active samples, respectively. In the inhibitory activity test, the optically active sample with a later retention time was used.

¹H-NMR (400 MHz, DMSO-d₆, δppm): 1.05-1.30 (2H, m), 1.40 (2H, m), 1.59 (2H, m), 2.08 (3H, s), 3.14 (2H, m), 3.52 (1H, m), 6.68 (1H, m), 7.04 (1H, m), 7.19 (2H, m), 7.37 (2H, m), 7.56-7.63 (2H, m), 7.71 (2H, m), 7.95 (1H, m), 8.29 (1H, s), 8.47 (1H, m), 11.64 (1H, s).

ESI-MS Found: m/z 403[M+H]⁺

### Reference Example 1

### 5-chloro-1-methyl-N-(2-methyldecahydroisoquinolin-6-yl)-1H-indole-2-carboxamide

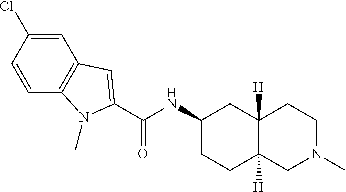

The compound obtained in Production example 5-4 (3.3 g) was dissolved in trifluoroacetic acid (30 mL), and the resulting solution was stirred at room temperature for 20 minutes. To the reaction solution, chloroform and a 5 N aqueous sodium hydroxide solution were added, and the resulting solution was stirred at room temperature for 5 minutes, and then, the chloroform layer was separated. The aqueous layer was extracted with chloroform, and the chloroform layer was combined with the previously obtained chloroform layer. The combined chloroform layer was dried over anhydrous sodium sulfate and concentrated under reduced pressure. The residue was dissolved in a 1% acetic acid-chloroform solution (100 mL), and formalin (0.66 mL) and sodium triacetoxy borohydride (1.9 g) were added thereto, and the resulting solution was stirred at room temperature for 3 hours. To the reaction solution, a 1 N sodium hydroxide solution was added, and the solution was extracted with chloroform. The chloroform layer was dried over anhydrous sodium sulfate and then concentrated under reduced pressure. The resulting residue was purified by silica gel column chromatography (chloroform:methanol (supplemented with 1% triethylamine)=80:1 to 20:1), whereby the title compound was obtained as a colorless oily substance. Diethyl ether was added to the colorless oily substance to suspend it, and a precipitated solid was collected by filtration and dried under reduced pressure, whereby the title compound (2.1 g) in the form of a white solid was obtained as a single optically active isomer (the arrangement in the structural formula is a relative configuration).

¹H-NMR (400 MHz, CDCl₃, δppm): 1.04-1.20 (2H, m), 1.37 (3H, m), 1.57 (2H, m), 1.69 (2H, m), 1.93 (3H, m), 2.28 (3H, s), 2.78 (1H, m), 2.91 (1H, m), 4.03 (3H, s), 4.41 (1H, m), 6.33 (1H, m), 6.76 (1H, br d, J=0.7 Hz), 7.29 (2H, m), 7.60 (1H, dd, J=1.8, 0.7 Hz).

ESI-MS Found: m/z 360[M+H]⁺

### INDUSTRIAL APPLICABILITY

It can be expected that the compounds according to the invention have usefulness in the prevention or treatment of various diseases related to QRFP43 or 26RFa, for example, cardiovascular diseases such as hypertension, arteriosclerosis, renal diseases, heart diseases and vasospasm; bulimia; and metabolic diseases such as obesity, diabetes, abnormal hormone secretion, hypercholesterolemia, hyperlipidemia, gout and fatty liver. Therefore, the compounds of the invention can be used as a preventive or remedy for pain, circadian rhythm disorder, atherosclerosis, obesity-related gastroesophageal reflux, obesity hypoventilation syndrome (Pickwickian syndrome), hypertriglyceridemia, low HDL cholesterolemia, cardiovascular diseases such as coronary heart diseases (CHD), peripheral vascular diseases and sudden death, pain, osteoporosis-related diseases, low back pain, anesthetic hypersensitivity and the like.

1-18. (canceled)

19. A compound represented by formula (I) or a pharmaceutically acceptable salt thereof: 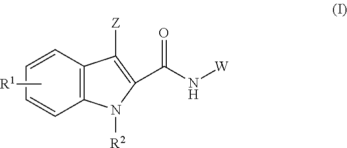
[wherein R¹ represents a hydrogen atom, halogen, C₁₋₆ alkyl, halo-C₁₋₆ alkyl, C₁₋₆ alkyloxy or halo-C₁₋₆ alkyloxy;
R² represents a hydrogen atom, C₁₋₆ alkyl or halo-C₁₋₆ alkyl;
W represents a group represented by the formula (w-1) or (w-2): 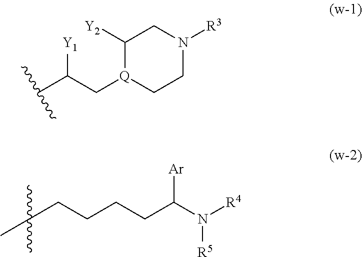
(wherein R³ represents C₁₋₆ alkyl which may be substituted by a substituent selected from a group consisting of halogen, hydroxy and phenyl;
Y₁ and Y₂ both represent a hydrogen atom or Y₁ and Y₂ together form —CH₂—CH₂—;
Q represents CH or N;
Ar is selected from a group consisting of phenyl, furyl, pyridyl, pyrimidyl, (1-methyl-1H-pyrazol)-3-yl and (1-methyl-1H-pyrazol)-4-yl;
R⁴ and R⁵ each independently represent a hydrogen atom or C₁₋₆ alkyl or R⁴ and R⁵, together with the nitrogen atom to which they are attached form a pyrrolidine ring or a piperidine ring);
Z represents aryl or heteroaryl (with the proviso that imidazolyl is excluded), wherein the aryl or heteroaryl may be substituted by one to three substituents selected from a group consisting of halogen, C₁₋₆ alkyl, halo-C₁₋₆ alkyl, C₁₋₆ alkyloxy, halo-C₁₋₆ alkyloxy, hydroxy, amino, mono-C₁₋₆ alkylamino, alkylamino, C₁₋₆ alkyloxycarbonyl, C₁₋₆ alkyloxycarbonylamino, C₁₋₆ alkyloxycarbonyl(C₁₋₆ alkyl) amino, C₁₋₆ alkylcarbonyl, C₁₋₆ alkylcarbonyloxy, C₁₋₆ alkylcarbonylamino, C₁₋₆ alkylcarbonyl(C₁₋₆ alkyl)amino, carbamoyl, mono-C₁₋₆ alkylcarbamoyl, di-C₁₋₆ alkylcarbamoyl, carbamoylamino, mono-C₁₋₆ alkylcarbamoylamino, alkylcarbamoylamino, mono-C₁₋₆ alkylcarbamoyl(C₁₋₆ alkyl) amino, di-C₁₋₆ alkylcarbamoyl(C₁₋₆ alkyl)amino, carbamoyloxy, mono-C₁₋₆ alkylcarbamoyloxy, alkylcarbamoyloxy, C₁₋₆ alkylsulfonyl, C₁₋₆ alkylsulfonylamino, C₁₋₆ alkylsulfonyl (C₁₋₆ alkyl)amino, sulfamoyl, mono-C₁₋₆ alkylsulfamoyl, alkylsulfamoyl, sulfamoylamino, mono-C₁₋₆ alkylsulfamoylamino, alkylsulfamoylamino, mono-C₁₋₆ alkylsulfamoyl(C₁₋₆ alkyl)amino and di-C₁₋₆ alkylsulfamoyl (C₁₋₆ alkyl)amino)].

20. The compound or the pharmaceutically acceptable salt thereof according to claim 19, wherein the compound is represented by a formula (I-1): 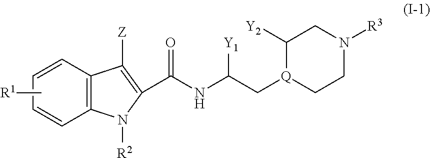
[wherein R¹, R², R³ , Y₁, Y₂, Q and Z have the same meanings as defined in claim 1].

21. The compound or the pharmaceutically acceptable salt thereof according to claim 20, wherein Y₁ and Y₂ are both hydrogen atom.

22. The compound or the pharmaceutically acceptable salt thereof according to any one of claims 21, wherein Z is phenyl, furyl, thienyl or thiazolyl.

23. The compound or the pharmaceutically acceptable salt thereof according to any one of claims 22, wherein R¹ is a hydrogen atom or chloro.

24. The compound or the pharmaceutically acceptable salt thereof according to claim 20, wherein Y₁ and Y₂ together form —CH₂—CH₂—.

25. The compound or the pharmaceutically acceptable salt thereof according to any one of claims 24, wherein Z is phenyl, furyl, thienyl or thiazolyl.

26. The compound or the pharmaceutically acceptable salt thereof according to any one of claims 25, wherein R¹ is a hydrogen atom or chloro.

27. The compound or the pharmaceutically acceptable salt thereof according to claim 1, wherein the compound is represented by a formula (I-2): 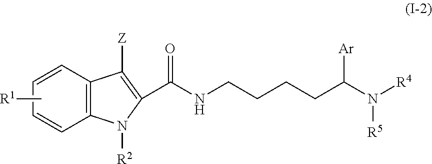
[wherein R¹, R², R⁴, R⁵, Z and Ar have the same meanings as defined in claim 1].

28. The compound or the pharmaceutically acceptable salt thereof according to any one of claims 27, wherein Z is phenyl, furyl, thienyl or thiazolyl.

29. The compound or the pharmaceutically acceptable salt thereof according to claim 28, wherein Ar is furyl or pyridyl.

30. The compound or the pharmaceutically acceptable salt thereof according to any one of claims 29, wherein R¹ is a hydrogen atom or chloro.

31. The compound or the pharmaceutically acceptable salt thereof according to claim 30, wherein R⁴ and R⁵ are each independently a hydrogen atom, methyl, ethyl, n-propyl or isopropyl.

32. The compound or the pharmaceutically acceptable salt thereof according to claim 30, wherein R⁴ and R⁵ together with the nitrogen atom to which they are attached form a pyrrolidine ring.

33. The compound or the pharmaceutically acceptable salt thereof according to claim 19, wherein the compound represented by the formula (I) is selected from a group consisting of:
5-chloro-3-(3-fury)-N-[2-(4-methylpiperazin-1-yl)ethyl]-1H-indole-2-carboxamide;
5-chloro-N-[2-(4-methylpiperazin-1-yl)ethyl]-3-(3-thienyl)-1H-indole-2-carboxamide;
5-chloro-N-[2-(4-methylpiperazin-1-yl)ethyl]-3-(2-thienyl)-1H-indole-2-carboxamide;
5-chloro-N-[2-(4-methylpiperazin-1-yl)ethyl]-3-(1,3-thiazol-4-yl)-1H-indole-2-carboxamide;
5-chloro-N-[2-(4-methylpiperazin-1-yl)ethyl]-3-phenyl-1H-indole-2-carboxamide;
5-chloro-3-(3-fury)-N-[2-(1-methylpiperidin-4-yl)ethyl]-1H-indole-2-carboxamide;
5-chloro-3-(3-fury)-N-(2-methyldecahydroisoquinolin-6-yl)-1H-indolecarboxamide;
5-chloro-3-(3-fury)-N-[5-(2-furyl)-5-pyrrolidin-1-ylpentyl]-1H-indole-2-carboxamide; and
3-(3-fury)-N-[5-(methylamino)-5-pyridin-2-ylpentyl]-1H-indole-2-carboxamide.

34. A pharmaceutical composition comprising the compound or the pharmaceutically acceptable salt thereof according to claim 19 and a pharmaceutically acceptable carrier.

35. A method for treating obesity in a subject in need thereof which comprises administering to the subject a therapeutically effective amount of a compound of claim 19, or a pharmaceutically acceptable salt thereof.
